(19) United States
(12) Patent Application Publication
(10) Pub. No.: US 2023/0048144 A1
(43) Pub. Date: Feb. 16, 2023

(54) HPV VACCINE

(71) Applicant: Merck Sharp & Dohme LLC, Rahway, NJ (US)

(72) Inventors: Andrew Bett, Lansdale, PA (US); Colleen M. Barr, Pennsburg, PA (US); John P. Bilello, San Carlos, CA (US); Pedro J. Cejas, Oreland, PA (US); Amy S. Espeseth, Chalfont, PA (US); Tong-Ming Fu, Philadelphia, PA (US); Erica L. Strable, Gilbertsville, PA (US); Sara Zimmermann, Levittown, PA (US)

(73) Assignee: Merck Sharp & Dohme LLC, Rahway, NJ (US)

(21) Appl. No.: 17/817,382

(22) Filed: Aug. 4, 2022

Related U.S. Application Data
(60) Provisional application No. 63/230,426, filed on Aug. 6, 2021.

(51) Int. Cl. A61K 39/39; A61K 39/12; A61P 31/20

(52) U.S. Cl. CPC A61K 39/39; A61K 39/12; A61P 31/20; A61K 2039/55583

(57) ABSTRACT

The present disclosure provides, among other things, a single-dose vaccine composition that includes a chitosan adjuvant and HPV virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, where the single-dose vaccine composition provides enhanced or comparable HPV vaccine response in comparison to a similar multiple-dose vaccine formulated without such chitosan adjuvant.

### CROSS REFERENCE TO RELATED APPLICATIONS

This application claims the benefit of priority to U.S. Provisional Application No. 63/230,426, filed Aug. 6, 2021, the disclosure of which is incorporated herein by its entirety.

### FIELD OF THE INVENTION

The present invention relates generally to the prevention of human papillomavirus (HPV) infection. More specifically, the invention relates to pharmaceutical compositions and formulations comprising virus-like particles (VLPs) of HPV and a chitosan adjuvant, which can be administered as a single-dose vaccine. The present disclosure provides, among other things, a single-dose vaccine composition that includes a chitosan adjuvant and an HPV vaccine, where a single administration of the vaccine composition provides a comparable or enhanced immune response in comparison to multiple administrations of the same HPV vaccine formulated without a chitosan adjuvant. Further provided are methods of using the disclosed compositions and formulations.

### BACKGROUND

Human papillomaviruses (HPVs) are small, double-stranded DNA viruses that infect the skin and internal squamous mucosal epithelia of men and women. HPVs are classified based on their carcinogenic properties. HPVs include major (L1) and minor (L2) capsid proteins. Over 200 distinct HPV genotypes have been identified (Li et al., “Rational design of a triple-type human papillomavirus vaccine by compromising viral-type specificity,” Nature, 9:5360 (2018)), many of which have been associated with pathologies ranging from benign proliferative warts to malignant carcinomas of the cervix (for review, see McMurray et al., Int. J. Exp. Pathol. 82(1): 15-33 (2001)). Those HPV types labeled as “high-risk” include 16, 18, 31, 33, 35, 39, 45, 51, 52, 56, 58, 68, and 59. (Chan et al., “Human Papillomavirus Infection and Cervical Cancer: Epidemiology, Screening, and Vaccination—Review of Current Perspectives,” Journal of Oncology, vol. 2019, Article ID 3257939, 2019.)

HPV is the primary etiological agent in cervical cancer, one of the most common cancer types in women, as well as squamous cell carcinomas of the anus, tonsil, tongue, vulva, vagina, and penis. HPV16 and HPV18 are well known as the most virulent of the high-risk HPV types as they cause approximately 70% of all invasive cervical cancer in the world.

Papillomaviruses are small (50-60 nm), nonenveloped, icosahedral DNA viruses that encode up to eight early (E1-E7) and two late (L1-L2) genes. The L1 protein is the major capsid protein and has a molecular weight of 55-60 kDa. Expression of the L1 protein or a combination of the L1 and L2 proteins in yeast, insect cells, mammalian cells or bacteria leads to self-assembly of virus-like particles (VLPs) (for review, see Schiller and Roden, in Papillomavirus Reviews: Current Research on Papillomaviruses; Lacey, ed. Leeds, UK: Leeds Medical Information, pp 101-12 (1996)).

VLPs are morphologically similar to authentic virions and are capable of inducing high titers of neutralizing antibodies upon administration into animals or humans. Because VLPs do not contain the potentially oncogenic viral genome, they present a safe alternative to the use of live virus in HPV vaccine development (for review, see Schiller and Hidesheim, J Clin. Virol. 19: 67-74 (2000)). For this reason, the L1 and L2 genes have been identified as immunological targets for the development of prophylactic and therapeutic vaccines for HPV infection and disease.

VLP-based vaccines have proven to be effective at inducing immune responses in human subjects vaccinated with bivalent HPV 16 and 18 (Harper et al. Lancet 364 (9447): 1757-65 (2004)), quadrivalent HPV 6, 11, 16, and 18 (Villa et al. Vaccine 24: 5571-5583 (2006)) and multi-valent HPV 6, 11, 16, 18, 31, 33, 45, 52 and 58 VLP-based vaccines. Three marketed VLP-based vaccines against HPV are administered according to 2 or 3 dose regimens. CERVARIX® (GlaxoSmithKline Biologicals, Rixensart, Belgium), is a bivalent vaccine protective against HPV 16 and 18. GARDASIL® and GARDASIL®9 (Merck & Co., Inc., Kenilworth, N.J., USA) protect against two and seven additional HPV types, respectively, and prevent additional HPV-related anogenital diseases, including wart formation. The additional five high-risk strains in GARDASIL®9 compared to GARDASIL® increase protection from about 70% of anogenital malignancies to about 90%. (Id., M. Nygård, et al., “Evaluation of the long-term anti-human papillomavirus 6 (HPV6), 11, 16, and 18 immune responses generated by the quadrivalent HPV vaccine,” Clinical and Vaccine Immunology, vol. 22, no. 8, pp. 943-948, 2015.)

Though improving, worldwide HPV vaccination rates remain suboptimal. The worldwide coverage of HPV vaccination rates can be improved by reducing the number of healthcare practitioner visits required for the vaccination, increasing education on HPV disease prophylaxis, and alleviating the social stigma associated with vaccination. The proportion of adolescents in the Americas and in Europe completing a two dose vaccination series is estimated to be under 50%. Accordingly, it is desirable to improve HPV vaccination rates by generating improved vaccines that can generate immunity against HPV through a single administration that provides a comparable immune response to existing HPV vaccines that require 2 or more doses.

There is a need for an HPV vaccine that can be administered as a single injection and provide comparable or enhanced initial anti-HPV immune response when compared to the standard multi-dose HPV vaccine.

### SUMMARY OF THE INVENTION

The present invention provides a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82, a chitosan; and a pharmaceutically acceptable carrier. In one aspect, the present invention also provides a pharmaceutical composition comprising an aluminum adjuvant, virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82, a chitosan adjuvant; and a pharmaceutically acceptable carrier.

The present invention further provides a pharmaceutical composition comprising virus-like particles (VLPs) of at least one type of human papillomavirus (HPV), wherein the at least one type of HPV is selected from the group consisting of HPV types: 6, 11, 16, 18, 26, 31, 33, 35, 39, 45, 51, 52, 53, 55, 56, 58, 59, 66, 68, 73, and 82; and 0.1 μg to about 50 mg of a chitosan, wherein the HPV VLPs comprise recombinant HPV L1 or recombinant HPV L1+L2 protein, wherein the HPV VLPs of each of the at least one HPV types are present in a concentration of about 10 μg to about 300 μg per 0.5 mL of the pharmaceutical composition, and wherein the total VLP concentration is between 10 μg and 2000 μg per 0.5 mL of the pharmaceutical composition. In one aspect, the present invention also provides a pharmaceutical composition comprising an aluminum adjuvant, virus-like particles (VLPs) of at least one type of human papillomavirus (HPV), wherein the at least one type of HPV is selected from the group consisting of HPV types: 6, 11, 16, 18, 26, 31, 33, 35, 39, 45, 51, 52, 53, 55, 56, 58, 59, 66, 68, 73, and 82; and 0.1 μg to about 50 mg of a chitosan, wherein the HPV VLPs comprise recombinant HPV L1 or recombinant HPV L1+L2 protein, wherein the HPV VLPs of each of the at least one HPV types are present in a concentration of about 10 μg to about 300 μg per 0.5 mL of the pharmaceutical composition, and wherein the total VLP concentration is between 10 μg and 2000 μg per 0.5 mL of the pharmaceutical composition.

The present invention further provides a single-dose vaccine composition that includes a chitosan, virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, and a pharmaceutically acceptable carrier; wherein the single-dose vaccine composition provides an elevated or comparable anti-HPV immune response relative to multiple doses of the same composition formulated without a chitosan. In one aspect, the present invention also provides a single-dose vaccine composition comprising an aluminum adjuvant, a chitosan adjuvant, virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, and a pharmaceutically acceptable carrier; wherein the single-dose vaccine composition provides an elevated or comparable anti-HPV immune response relative to multiple doses of the same composition formulated without a chitosan.

The present invention also provides a method of inducing an immune response to a human papillomavirus (HPV) in a human patient comprising administering to the patient a pharmaceutical composition comprising at least one virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82, a chitosan; and a pharmaceutically acceptable carrier, and optionally an aluminum adjuvant.

The present invention also provides a method of inducing an immune response to a human papillomavirus (HPV) in a human patient comprising administering to the patient a pharmaceutical composition comprising virus-like particles (VLPs) of at least one type of human papillomavirus (HPV), wherein the at least one type of HPV is selected from the group consisting of HPV types: 6, 11, 16, 18, 26, 31, 33, 35, 39, 45, 51, 52, 53, 55, 56, 58, 59, 66, 68, 73, and 82; and 0.1 μg to about 50 mg of a chitosan, and optionally an aluminum adjuvant, wherein the HPV VLPs comprise recombinant HPV L1 or recombinant HPV L1+L2 protein, wherein the HPV VLPs of each of the at least one HPV types are present in a concentration of about 10 μg to about 300 μg per 0.5 mL of the pharmaceutical composition, and wherein the total VLP concentration is between 10 μg and 2000 μg per 0.5 mL of the pharmaceutical composition.

The present invention also provides a method of inducing an immune response to a human papillomavirus (HPV) in a human patient comprising administering to the patient a single-dose vaccine composition that includes a chitosan adjuvant, virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, and a pharmaceutically acceptable carrier, and optionally an aluminum adjuvant; wherein the single-dose vaccine composition provides an elevated or comparable anti-HPV immune response relative to multiple doses of the same composition formulated without a chitosan.

The present invention also provides a method of inducing an immune response to a human papillomavirus (HPV) in a human patient comprising co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan. The present invention also provides a method of inducing an immune response to a human papillomavirus (HPV) in a human patient comprising co-administering to the patient (a) a pharmaceutical composition comprising an aluminum adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan.

The present invention also provides a method of preventing infection of a human patient by a human papillomavirus (HPV) comprising administering to the patient a pharmaceutical composition comprising at least one virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82, a chitosan; and a pharmaceutically acceptable carrier, and optionally an aluminum adjuvant.

The present invention also provides a method of preventing infection of or reducing the likelihood of infection of a human patient by a human papillomavirus (HPV) comprising administering to the patient a pharmaceutical composition comprising virus-like particles (VLPs) of at least one type of human papillomavirus (HPV), wherein the at least one type of HPV is selected from the group consisting of HPV types: 6, 11, 16, 18, 26, 31, 33, 35, 39, 45, 51, 52, 53, 55, 56, 58, 59, 66, 68, 73, and 82; and 0.1 μg to about 50 mg of a chitosan, and optionally an aluminum adjuvant, wherein the HPV VLPs comprise recombinant HPV L1 or recombinant HPV L1+L2 protein, wherein the HPV VLPs of each of the at least one HPV types are present in a concentration of about 10 μg to about 300 μg per 0.5 mL of the pharmaceutical composition, and wherein the total VLP concentration is between 10 μg and 2000 μg per 0.5 mL of the pharmaceutical composition. In one embodiment, the HPV VLPs do not comprise HPV L2 protein.

The present invention also provides a method of preventing infection of or reducing the likelihood of infection of a human patient by a human papillomavirus (HPV) comprising administering to the patient a single-dose vaccine composition that includes (a) a chitosan, (b) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, (c) a pharmaceutically acceptable carrier, and (d) optionally an aluminum adjuvant; wherein the single-dose vaccine composition provides an elevated or comparable anti-HPV immune response relative to multiple doses of the same composition formulated without a chitosan.

The present invention also provides a method of preventing infection of or reducing the likelihood of infection of a human patient by a human papillomavirus (HPV) comprising co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan. The present invention also provides a method of preventing infection of a human patient by a human papillomavirus (HPV) comprising co-administering to the patient (a) a pharmaceutical composition comprising an aluminum adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan.

The present invention also provides a kit comprising: (a) a human papilloma virus (HPV) vaccine; and (b) a chitosan, and an optionally an aluminum adjuvant.

The present invention provides a method of delivering a pharmaceutical composition to a subject that induces a neutralizing titer against an HPV antigen in the subject comprising: administering to the subject a pharmaceutical composition comprising: a chitosan, and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, whereby the administration of the pharmaceutical composition induces a neutralizing titer against the HPV antigen in the subject, wherein a single dose of the pharmaceutical composition provides enhanced or comparable neutralizing titers when compared to multiple doses of the same pharmaceutical composition when the same composition is formulated without chitosan. The present invention also provides a method of delivering a pharmaceutical composition to a subject that induces a neutralizing titer against an HPV antigen in the subject comprising: administering to the subject a pharmaceutical composition comprising: a chitosan adjuvant, and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, and optionally an aluminum adjuvant, whereby the administration of the pharmaceutical composition induces a neutralizing titer against the HPV antigen in the subject, wherein a single dose of the pharmaceutical composition provides enhanced or comparable neutralizing titers when compared to multiple doses of the same pharmaceutical composition when the same composition is formulated without chitosan.

### Definitions

As used throughout the specification and in the appended claims, the singular forms “a,” “an,” and “the” include the plural reference unless the context clearly dictates otherwise.

As used throughout the specification and appended claims, the following definitions and abbreviations apply:

AAHS: As used herein, the term “AAHS” refers to an amorphous aluminum hydroxyphosphate sulfate adjuvant.

About: As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. For example, in some embodiments, the term “about” can encompass a range of values within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Acid-Soluble Chitosan: As used herein, the term “acid-soluble chitosan” refers to chitosan that is prepared by solubilizing a chitosan powder in an acid. Examples of acid-soluble chitosan include non-salt or free base forms of chitosan, e.g., Sigma-Aldrich Product Number 448869.

Adjuvant: As used herein, the term “adjuvant” refers to a composition or compound that is capable of enhancing the immune response against an antigen of interest. Adjuvants are substances or combinations of substances that are used in conjunction with a vaccine antigen to enhance (e.g., increase, accelerate, prolong and/or possibly target) the specific immune response to the vaccine antigen or modulate to a different type (e.g., switch a Th1 immune response to a Th2 response, or a humoral response to a cytotoxic T cell response) in order to enhance the clinical effectiveness of the vaccine. In some embodiments, the adjuvant may modify (Th1/Th2) the immune response. In some embodiments, the adjuvant may boost the strength and longevity of the immune response. In some embodiments, the adjuvant may broaden the immune response to a concomitantly administered antigen. In some embodiments, the adjuvant may be capable of inducing strong antibody and T cell responses. In some embodiments, the adjuvant may be capable of increasing the polyclonal ability of the induced antibodies. In some embodiments, the adjuvant may be used to decrease the amount of antigen necessary to provoke the desired immune response and provide protection against the disease. In some embodiments, the adjuvant may be used to decrease the number of injections needed in a clinical regimen to induce a durable immune response and provide protection against the disease. Adjuvant containing formulations described herein may demonstrate enhancements in humoral and/or cellular immunogenicity of vaccine antigens, for example, subunit vaccine antigens. Adjuvants of the present invention are not used to deliver antigens, antibodies, active pharmaceutical ingredients (APIs), or VLPs.

Administration: As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscularly (IM), intradermally (ID) etc.) and the like.

Agent: As used herein, the term “agent” refers to a particle, compound, molecule, or entity of any chemical class including, for example, a VLP, a small molecule, polypeptide (e.g., a protein), polynucleotide (e.g., a DNA polynucleotide or an RNA polynucleotide), saccharide, lipid, or a combination or complex thereof. In some embodiments, the term “agent” can refer to a compound, molecule, or entity that includes a polymer, or a plurality thereof.

Antibody: As used herein, the term “antibody” (or “Ab”) refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies

Antigen: As used herein, the term “antigen” refers to any antigen that can generate one or more immune responses. The antigen may be a protein (including recombinant proteins), VLP, polypeptide, or peptide (including synthetic peptides). The antigen may be one that generates a humoral and/or CTL immune response.

API: As used herein, the term “API” refers to an active pharmaceutical ingredient, e.g., HPV VLPs, which is a component of the compositions or formulations disclosed herein that is biologically active (e.g. capable of inducing an appropriate immune response) and confers a therapeutic or prophylactic benefit to a person or animal in need thereof. As used herein, an API is a vaccine active ingredient.

Chitosan: As used herein, the term “chitosan” refers to a polysaccharide containing randomly distributed β-(1→4)-linked D-glucosamine (deacetylated unit) and N-acetyl-D-glucosamine (acetylated unit) (i.e. α-(1-4)-2-amino-2-deoxy-β-d-glucan), which is mostly deacetylated). Chitosan may be isolated after chemical modification of crustacean chitin shells or other natural sources like fungi. Alternatively, chitosan may be generated by a chemically synthetic route. Chitosan could be further modified by various degrees of acetylation, alkylation, chain length and the addition of other chemical modifications, such as adding thiols, amines, and other functional groups. As used herein, chitosan refers to a class of molecules having a degree of deacetylation above 75%. The term “chitosan” as used herein includes acid-soluble chitosan and water-soluble chitosan.

Chitosan Adjuvant: As used herein, the term “chitosan adjuvant” refers to a composition comprising a chitosan or a chitosan compound that is capable of enhancing the immune response against an antigen of interest.

Co-administration: As used herein, the term “co-administration” or “co-administering” in relation to the chitosan adjuvant and a pharmaceutical formulation (e.g., an HPV vaccine) refers to administration of a chitosan adjuvant and a pharmaceutical formulation (e.g., an HPV vaccine) concurrently, i.e., simultaneously in time, or sequentially, i.e., administration of an HPV vaccine followed by administration of the chitosan adjuvant (or vice versa). That is, after administration of the HPV vaccine (or chitosan adjuvant), the chitosan adjuvant (or HPV vaccine) can be administered substantially immediately after the HPV vaccine (or chitosan adjuvant) or the chitosan adjuvant (or the HPV vaccine) can be administered after an effective time period after the HPV vaccine (or chitosan adjuvant); the effective time period is the amount of time period is generally within 1, 2, 3, 5, 10, 15, 20, 25, 30, 45, or 60 minutes.

Deacetylation: As used herein, the term “deacetylation” refers to the removal of an acetyl group from an organic compound. The deacetylation of solids may be measured via methods known in the art, such as, NMR, UV (EP method), or IR. In addition, the deacetylation of a composition in solution may be measured via methods known in the art, such as, CZE (capillary zone electrophoresis), GC-MS, ion chromatography, and SEC-UV.

Dose: As used herein, the term “dose” means a quantity of an agent, API, formulation, or pharmaceutical composition administered or recommended to be administered at a particular time.

HPV and PV: As used herein, the terms “HPV” and “PV” refer to human papillomavirus and papillomavirus, respectively.

Multiple-dose: As used herein, the term “multiple-dose” refers to a vaccine composition, or pharmaceutical composition, that requires more than one dose or administration or injection of the components therein in a clinical regimen to induce a durable immune response and provide protection from a disease. One of skill in the art would understand how to determine a durable immune response, e.g., by measuring antibody titers over a specified period of time.

Patient: As used herein, the term “patient” refers to any human being that is to receive the HPV vaccines, or pharmaceutical compositions, described herein. As defined herein, “patient” includes those already infected with one or more types of HPV as well as those in which infection with one or more types of HPV is to be prevented.

Pharmaceutically acceptable: As used herein with respect to a carrier, diluent, or excipient of a pharmaceutical composition, the term “pharmaceutically acceptable” indicates that a carrier, diluent, or excipient must be compatible with the other ingredients of the composition and not deleterious to the recipient thereof.

Pharmaceutical composition: As used herein, the term “pharmaceutical composition,” refers to a composition containing an active pharmaceutical or biological ingredient, along with one or more additional components, e.g., a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. The pharmaceutical compositions or formulations can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed. In some embodiments, a pharmaceutical composition can be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, the term formulation refers to a single-dose of vaccine, which can be included in any volume suitable for injection.

Single-dose: As used herein, the term “single-dose” refers to a vaccine composition that only requires one administration or injection in a clinical regimen to induce a durable immune response and provide protection from a disease. One of skill in the art would understand how to determine a durable immune response, e.g., by measuring antibody titers over a specified period of time.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutically Effective Amount: As used herein, the term “therapeutically effective amount” refers to an amount of the active ingredient (e.g. therapeutic protein, vaccine, or antibody) sufficient to produce the desired therapeutic effect in a human or animal, e.g., the amount necessary to elicit an immune response, treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given antibody or therapeutic protein or vaccine antigen.

Vaccine: As used herein, the term “vaccine” or “vaccine composition” refers to a substance or preparation used to stimulate the production of antibodies and provide immunity against one or several diseases, prepared from the causative agent of a disease, its products, or a synthetic substitute, treated to act as an antigen without inducing the disease. A vaccine composition may include at least one antigen or VLP in a pharmaceutically acceptable vehicle useful for inducing an immune response in a subject. The vaccine composition is administered by doses and techniques known to those skilled in the pharmaceutical or veterinary fields, taking into account factors such as the age, sex, weight, species, and condition of the recipient animal and the route of administration.

Valent: As used herein, the term “valent” refers to the presence of a specified number of antigens in a vaccine. For example, the terms bi-valent, bivalent, 2 valent, or 2-valent refer to two different antigens. Similarly, the terms quadrivalent, 4 valent, or 4-valent refer to four different antigens and the terms nonavalent, 9 valent or 9-valent refer to nine different antigens.

Viscosity: As used herein, viscosity refers to the measure of a substance's resistance to deformation or flow at a given rate. Viscosity can be measured, for example, by using a viscometer at a given shear rate or shear rates that are appropriately selected by those skilled in the art to accurately measure viscosity in the viscosity range of the sample of interest. The viscosity of the substances used herein were measured using a viscometer (e.g. Brookfield DVII+pro) at 20° C. at a standard concentration (e.g. 1% in 1% acetic acid). In some embodiments, the viscosity is a measure of a solid, such as e.g. chitosan, dissolved in a 1% acetic acid solution to achieve a final chitosan concentration of 1% (w/v).

Virus Like Particles: As used herein, the term “virus like particles” or “VLPs” refers to agents that are morphologically similar to authentic virions or provide an arrayed display of an antigen and are capable of inducing high antibody neutralization ratings after administration in an animal. VLPs lack the viral genetic material of the authentic virions and are thus non-infectious.

Water-Soluble Chitosan: As used herein, the term “water-soluble chitosan” refers to chitosan that is prepared by solubilizing a chitosan powder in water or aqueous buffer. Examples of water-soluble chitosan include chitosan hydrochloride, chitosan chloride, chitosan ascorbate, carboxylic acid salts of chitosan, and the like, e.g., Heppe Medical Chitosan Item Numbers 54046 and 54047.

### BRIEF DESCRIPTION OF THE DRAWINGS

FIG. 1A shows a graphical depiction of HPV VLP 16 antibody levels in rabbits after a single inoculation of a 9 valent HPV vaccine with and without a chitosan adjuvant and a multiple-inoculation of a 9 valent HPV vaccine without a chitosan adjuvant.

FIG. 1B shows a graphical depiction of VLP 18 antibody levels in rabbits after a single inoculation of a 9 valent HPV vaccine with and without a chitosan adjuvant and a multiple-inoculation of a 9 valent HPV vaccine without a chitosan adjuvant.

FIG. 2 shows a graphical depiction of serotype-specific HPV VLP antibody levels in rabbits measured at 48 weeks after a single inoculation of a 9 valent HPV vaccine combined with a chitosan adjuvant and multiple-inoculation of a 9 valent HPV vaccine without a chitosan adjuvant.

FIG. 3A shows a graphical depiction of HPV VLP 16 antibody levels in rhesus macaques after a single inoculation of a 9 valent HPV vaccine combined with a chitosan adjuvant and multiple-inoculation of a 9 valent HPV vaccine without a chitosan adjuvant.

FIG. 3B shows a graphical depiction of HPV VLP 18 antibody levels in rhesus macaques after a single inoculation of a 9 valent HPV vaccine combined with a chitosan adjuvant and multiple-inoculation of a 9 valent HPV vaccine without a chitosan adjuvant.

FIG. 4 shows a graphical depiction of individual HPV VLP antibody levels in rhesus macaques measured at 36 weeks after a single inoculation of a 9 valent HPV vaccine combined with a chitosan adjuvant and multiple-inoculation of a 9 valent HPV vaccine without a chitosan adjuvant.

FIG. 5A shows a graphical depiction of HPV VLP 16 antibody levels in rhesus macaques after a single inoculation of a 9 valent HPV vaccine combined with a chitosan adjuvant and multiple-inoculation of a 9 valent HPV vaccine without a chitosan adjuvant.

FIG. 5B shows a graphical depiction of HPV VLP 18 antibody levels in rhesus macaques after a single inoculation of a 9 valent HPV vaccine combined with a chitosan adjuvant and multiple-inoculation of a 9 valent HPV vaccine without a chitosan adjuvant.

FIG. 5C shows a graphical depiction of HPV VLP 16 antibody levels in rhesus macaques after a single inoculation of a 9 valent HPV vaccine combined with a chitosan adjuvant and multiple-inoculation of a 9 valent HPV vaccine without a chitosan adjuvant.

FIG. 5D shows a graphical depiction of HPV VLP 18 antibody levels in rhesus macaques after a single inoculation of a 9 valent HPV vaccine combined with a chitosan adjuvant and multiple-inoculation of a 9 valent HPV vaccine without a chitosan adjuvant.

FIG. 6 shows serotype-specific HPV VLP antibody levels in rhesus macaques measured at 6 weeks after a single inoculation of a 9 valent HPV vaccine combined with a chitosan adjuvant and multiple-inoculation of a 9 valent HPV vaccine without a chitosan adjuvant.

### DETAILED DESCRIPTION

Currently, there are multiple approved HPV vaccines that are composed of VLPs and are highly effective at protecting vaccinated patients against premalignant lesions, anogenital cancers and genital warts when administered prior to natural infection in subjects 9 years and older as multidose regimens. In accordance with this invention, it has been shown that a single-dose HPV vaccine composition that includes HPV VLPs of at least one HPV type (“targeted HPV types”) and a chitosan adjuvant are able to provide comparable or enhanced antibody titers to the same targeted HPV types when compared to multiple-doses of vaccine compositions that include VLPs of the targeted HPV types formulated, or administered, without a chitosan adjuvant. The compositions of the present invention, which comprise HPV VLPs and a chitosan adjuvant, are intended to generate immunity against HPV subtypes through a single-injection regimen that is comparable to, at least, a 2-3 injection regimen of such HPV vaccine, including an approved two-, four-, or nine-valent HPV vaccine which do not contain a chitosan adjuvant. It was surprisingly found that a single intramuscular injection of a chitosan adjuvant combined with an HPV vaccine provided a comparable or enhanced initial anti-HPV immune response when compared to the standard multi-dose protocol of known aluminum adjuvant-containing multivalent HPV vaccine.

### Chitosan Adjuvant

Chitin, a polymer of N-acetylglucosamine (i.e., (1-4)-2-acetamido-2-deoxyβ-d-glucan), is a significant component of the body of all crustaceans and is also present in the exoskeleton and the cell wall of fungi, insects, and yeast. Chitosan, i.e., α-(1-4)-2-amino-2-deoxy-β-d-glucan, is the mostly deacetylated form of the naturally occurring polysaccharide chitin. Chitosan is typically formed by deacetylation of chitin in the presence of alkali. The term “chitosan” refers to the class of molecules having a degree of deacetylation that is different from chitin. For example, molecules having a deacetylation below 75% are consider chitin. In contrast, molecules having a deacetylation above 75% are considered chitosan. The deacetylation of chitosan powder may be measured via NMR, UV (EP method), or IR. The deacetylation of chitosan in solution may be measured via CZE (capillary zone electrophoresis), GC-MS, ion chromatography, and SEC-UV.

In some embodiments, the chitosan adjuvant may include a chitosan. In some embodiments, the chitosan adjuvant may include a chitosan having a deacetylation of 75% or greater. In some embodiments, the chitosan adjuvant may include chitosan that has a degree of deacetylation in the range of about 75-99%. In some embodiments, the chitosan adjuvant may include chitosan that has a degree of deacetylation in the range of about 85-99%. In some embodiments, the chitosan adjuvant may include chitosan that has a degree of deacetylation greater than 90%. In some embodiments, the chitosan adjuvant may include chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. Deacetylation may be calculated according to any of the methods described herein.

In some embodiments, the chitosan adjuvant may include chitosan that is a water-soluble chitosan having a degree of deacetylation in the range of about 75-99%. In some embodiments, the chitosan adjuvant may include water-soluble chitosan that has a degree of deacetylation in the range of about 75-99%. In some embodiments, the chitosan adjuvant may include water-soluble chitosan that has a degree of deacetylation in the range of about 85-99%. In some embodiments, the chitosan adjuvant may include water-soluble chitosan that has a degree of deacetylation greater than 90%. In some embodiments, the chitosan adjuvant may include a water-soluble chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. In some embodiments, the water-soluble chitosan is chitosan hydrochloride. Deacetylation may be calculated according to any of the methods described herein.

In some embodiments, the chitosan adjuvant may include an acid-soluble chitosan that has a degree of deacetylation in the range of about 75-99%. In some embodiments, the acid-soluble chitosan is chitosan hydrochloride. In some embodiments, the chitosan adjuvant may include an acid-soluble chitosan that has a degree of deacetylation in the range of about 85-99%. In some embodiments, the chitosan adjuvant may include an acid-soluble chitosan that has a degree of deacetylation greater than 90%. In some embodiments, the chitosan adjuvant may include an acid-soluble chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. Deacetylation may be calculated according to any of the methods described above.

In some embodiments, the chitosan adjuvant includes a chitosan having a viscosity in the range of about 1 cP to about 200 cP when measured with a viscosimeter (e.g. Brookfield DVII+pro) at 20° C. at a standard concentration (e.g. 1% in 1% acetic acid or the viscosity is a measure of chitosan dissolved in a 1% acetic acid solution to achieve a final chitosan concentration of 1% (w/v)). In some embodiments, the chitosan has viscosity in the range of about 1 cP to about 100 cP. In some embodiments, the chitosan has viscosity in the range of about 5 cP to about 100 cP. In some embodiments, the chitosan has viscosity in the range of about 1 cP to about 50 cP. In some embodiments, the chitosan has viscosity in the range of about 5 cP to about 50 cP. In some embodiments, the chitosan has viscosity in the range of about 1 cP to about 25 cP. In some embodiments, the chitosan has viscosity in the range of about 5 cP to about 25 cP. In some embodiments, the chitosan has viscosity in the range of about 1 cP to about 20 cP. In some embodiments, the chitosan has viscosity in the range of about 5 cP to about 20 cP. In some embodiments, the chitosan has viscosity in the range of about 1 cP to about 15 cP. In some embodiments, the chitosan has viscosity in the range of about 5 cP to about 15 cP. In some embodiments, the chitosan has viscosity in the range of about 1 cP to about 10 cP. In some embodiments, the chitosan has viscosity in the range of about 5 cP to about 10 cP. In some embodiments, the chitosan has a viscosity less than 100. In some embodiments, the chitosan has a viscosity less than 75. In some embodiments, the chitosan has a viscosity less than 50. In some embodiments, the chitosan has a viscosity less than 40. In some embodiments, the chitosan has a viscosity less than 30. In some embodiments, the chitosan has a viscosity less than 20. In some embodiments, the chitosan has a viscosity less than 15. In some embodiments, the chitosan has a viscosity less than 10. In some embodiments, the chitosan has a viscosity less than 5. In some embodiments, the chitosan has viscosity of 1 cP, 2 cP, 3 cP, 4 cP, 5 cp, 6 cp, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan adjuvant includes a water-soluble chitosan having a viscosity in the range of about 1 cP to about 200 cP. In some embodiments, the water-soluble chitosan includes chitosan hydrochloride having a viscosity in the range of about 1 cP to about 200 cP when measured with a viscosimeter (e.g. Brookfield DVII+pro) at 20° C. at a standard concentration (e.g. 1% in 1% acetic acid or the viscosity is a measure of chitosan dissolved in a 1% acetic acid solution to achieve a final chitosan concentration of 1% (w/v)). In some embodiments, the chitosan hydrochloride has viscosity in the range of about 1 cP to about 100 cP. In some embodiments, the chitosan hydrochloride has viscosity in the range of about 5 cP to about 100 cP. In some embodiments, the chitosan hydrochloride has viscosity in the range of about 1 cP to about 50 cP. In some embodiments, the chitosan hydrochloride has viscosity in the range of about 5 cP to about 50 cP. In some embodiments, the chitosan hydrochloride has viscosity in the range of about 1 cP to about 25 cP. In some embodiments, the chitosan hydrochloride has viscosity in the range of about 5 cP to about 25 cP. In some embodiments, the chitosan hydrochloride has viscosity in the range of about 1 cP to about 20 cP. In some embodiments, the chitosan hydrochloride has viscosity in the range of about 5 cP to about 20 cP. In some embodiments, the chitosan hydrochloride has viscosity in the range of about 1 cP to about 15 cP. In some embodiments, the chitosan hydrochloride has viscosity in the range of about 5 cP to about 15 cP. In some embodiments, the chitosan hydrochloride has viscosity in the range of about 1 cP to about 10 cP. In some embodiments, the chitosan hydrochloride has viscosity in the range of about 5 cP to about 10 cP. In some embodiments, the chitosan hydrochloride has a viscosity less than 100. In some embodiments, the chitosan hydrochloride has a viscosity less than 75. In some embodiments, the chitosan hydrochloride has a viscosity less than 50. In some embodiments, the chitosan hydrochloride has a viscosity less than 40. In some embodiments, the chitosan hydrochloride has a viscosity less than 30. In some embodiments, the chitosan hydrochloride has a viscosity less than 20. In some embodiments, the chitosan hydrochloride has a viscosity less than 15. In some embodiments, the chitosan hydrochloride has a viscosity less than 10. In some embodiments, the chitosan hydrochloride has a viscosity less than 5. In some embodiments, the chitosan hydrochloride has viscosity of 1 cP, 2 cP, 3 cP, 4 cP, 5 cp, 6 cp, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan adjuvant includes an acid-soluble chitosan having a viscosity in the range of about 1 cP to about 200 cP when measured with a viscosimeter (e.g. Brookfield DVII+pro) at 20° C. at a standard concentration (e.g. 1% in 1% acetic acid). In some embodiments, the acid-soluble chitosan has viscosity in the range of about 1 cP to about 100 cP. In some embodiments, the acid-soluble chitosan has viscosity in the range of about 5 cP to about 100 cP. In some embodiments, the acid-soluble chitosan has viscosity in the range of about 1 cP to about 50 cP. In some embodiments, the acid-soluble chitosan has viscosity in the range of about 5 cP to about 50 cP. In some embodiments, the acid-soluble chitosan has viscosity in the range of about 1 cP to about 25 cP. In some embodiments, the acid-soluble chitosan has viscosity in the range of about 5 cP to about 25 cP. In some embodiments, the acid-soluble chitosan has viscosity in the range of about 1 cP to about 20 cP. In some embodiments, the acid-soluble chitosan has viscosity in the range of about 5 cP to about 20 cP. In some embodiments, the acid-soluble chitosan has viscosity in the range of about 1 cP to about 15 cP. In some embodiments, the acid-soluble chitosan has viscosity in the range of about 5 cP to about 15 cP. In some embodiments, the acid-soluble chitosan has viscosity in the range of about 1 cP to about 10 cP. In some embodiments, the acid-soluble chitosan has viscosity in the range of about 5 cP to about 10 cP. In some embodiments, the acid-soluble chitosan has a viscosity less than 100. In some embodiments, the acid-soluble chitosan has a viscosity less than 75. In some embodiments, the acid-soluble chitosan has a viscosity less than 50. In some embodiments, the acid-soluble chitosan has a viscosity less than 40. In some embodiments, the acid-soluble chitosan has a viscosity less than 30. In some embodiments, the acid-soluble chitosan has a viscosity less than 20. In some embodiments, the acid-soluble chitosan has a viscosity less than 15. In some embodiments, the acid-soluble chitosan has a viscosity less than 10. In some embodiments, the acid-soluble chitosan has a viscosity less than 5. In some embodiments, the acid-soluble chitosan has viscosity of 1 cP, 2 cP, 3 cP, 4 cP, 5 cp, 6 cp, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan adjuvant includes a water-soluble chitosan having a degree of deacetylation greater than 75% and a viscosity of less than 50 cP. In some embodiments, the water-soluble chitosan includes chitosan hydrochloride having a degree of deacetylation greater than 75% and a viscosity of less than 50 cP. In some embodiments, the chitosan adjuvant includes chitosan hydrochloride having a degree of deacetylation greater than 75% and a viscosity of less than 40 cP. In some embodiments, the chitosan hydrochloride has a degree of deacetylation greater than 75% and a viscosity of less than 30 cP. In some embodiments, the chitosan hydrochloride has a degree of deacetylation greater than 75% and a viscosity of less than 20 cP. In some embodiments, the chitosan hydrochloride has a degree of deacetylation greater than 75% and a viscosity of less than 10 cP. In some embodiments, the chitosan hydrochloride has a degree of deacetylation greater than 75% and a viscosity of less than 5 cP. In some embodiments, the chitosan hydrochloride has a degree of deacetylation greater than 85% and a viscosity of less than 50 cP. In some embodiments, the chitosan hydrochloride has a degree of deacetylation greater than 85% and a viscosity of less than 40 cP. In some embodiments, the chitosan hydrochloride has a degree of deacetylation greater than 85% and a viscosity of less than 30 cP. In some embodiments, the chitosan hydrochloride has a degree of deacetylation greater than 85% and a viscosity of less than 20 cP. In some embodiments, the chitosan hydrochloride has a degree of deacetylation greater than 85% and a viscosity of less than 10 cP. In some embodiments, the chitosan hydrochloride has a degree of deacetylation greater than 85% and a viscosity of less than 5 cP. In some embodiments, the chitosan hydrochloride has a degree of deacetylation greater than 90% and a viscosity of less than 50 cP. In some embodiments, the chitosan hydrochloride has a degree of deacetylation greater than 90% and a viscosity of less than 40 cP. In some embodiments, the chitosan hydrochloride has a degree of deacetylation greater than 90% and a viscosity of less than 30 cP. In some embodiments, the chitosan hydrochloride has a degree of deacetylation greater than 90% and a viscosity of less than 20 cP. In some embodiments, the chitosan hydrochloride has a degree of deacetylation greater than 90% and a viscosity of less than 10 cP. In some embodiments, the chitosan hydrochloride has a degree of deacetylation greater than 90% and a viscosity of less than 5 cP. In some embodiments, the chitosan hydrochloride has a degree of deacetylation greater than 95% and a viscosity of less than 50 cP. In some embodiments, the chitosan hydrochloride has a degree of deacetylation greater than 95% and a viscosity of less than 40 cP. In some embodiments, the chitosan hydrochloride has a degree of deacetylation greater than 95% and a viscosity of less than 30 cP. In some embodiments, the chitosan hydrochloride has a degree of deacetylation greater than 95% and a viscosity of less than 20 cP. In some embodiments, the chitosan hydrochloride has a degree of deacetylation greater than 95% and a viscosity of less than 10 cP. In some embodiments, the chitosan hydrochloride has a degree of deacetylation greater than 95% and a viscosity of less than 5 cP.

In some embodiments, the chitosan adjuvant includes an acid-soluble chitosan having a degree of deacetylation greater than 75% and a viscosity of less than 50 cP. In some embodiments, the acid-soluble chitosan has a degree of deacetylation greater than 75% and a viscosity of less than 40 cP. In some embodiments, the acid-soluble chitosan has a degree of deacetylation greater than 75% and a viscosity of less than 30 cP. In some embodiments, the acid-soluble chitosan has a degree of deacetylation greater than 75% and a viscosity of less than 20 cP. In some embodiments, the acid-soluble chitosan has a degree of deacetylation greater than 75% and a viscosity of less than 10 cP. In some embodiments, the acid-soluble chitosan has a degree of deacetylation greater than 75% and a viscosity of less than 5 cP. In some embodiments, the acid-soluble chitosan has a degree of deacetylation greater than 85% and a viscosity of less than 50 cP. In some embodiments, the acid-soluble chitosan has a degree of deacetylation greater than 85% and a viscosity of less than 40 cP. In some embodiments, the acid-soluble chitosan has a degree of deacetylation greater than 85% and a viscosity of less than 30 cP. In some embodiments, the acid-soluble chitosan has a degree of deacetylation greater than 85% and a viscosity of less than 20 cP. In some embodiments, the acid-soluble chitosan has a degree of deacetylation greater than 85% and a viscosity of less than 10 cP. In some embodiments, the acid-soluble chitosan has a degree of deacetylation greater than 85% and a viscosity of less than 5 cP. In some embodiments, the acid-soluble chitosan has a degree of deacetylation greater than 90% and a viscosity of less than 50 cP. In some embodiments, the acid-soluble chitosan has a degree of deacetylation greater than 90% and a viscosity of less than 40 cP. In some embodiments, the acid-soluble chitosan has a degree of deacetylation greater than 90% and a viscosity of less than 30 cP. In some embodiments, the acid-soluble chitosan has a degree of deacetylation greater than 90% and a viscosity of less than 20 cP. In some embodiments, the acid-soluble chitosan has a degree of deacetylation greater than 90% and a viscosity of less than 10 cP. In some embodiments, the acid-soluble chitosan has a degree of deacetylation greater than 90% and a viscosity of less than 5 cP. In some embodiments, the acid-soluble chitosan has a degree of deacetylation greater than 95% and a viscosity of less than 50 cP. In some embodiments, the acid-soluble chitosan has a degree of deacetylation greater than 95% and a viscosity of less than 40 cP.

In some embodiments, the acid-soluble chitosan has a degree of deacetylation greater than 95% and a viscosity of less than 30 cP. In some embodiments, the acid-soluble chitosan has a degree of deacetylation greater than 95% and a viscosity of less than 20 cP. In some embodiments, the acid-soluble chitosan has a degree of deacetylation greater than 95% and a viscosity of less than 10 cP. In some embodiments, the acid-soluble chitosan has a degree of deacetylation greater than 95% and a viscosity of less than 5 cP.

In some embodiments, the chitosan adjuvant includes chitosan in the amount of about 0.1 μg to about 200 mg. In some embodiments, the chitosan adjuvant includes chitosan in the amount of about 0.1 μg to about 100 mg. In some embodiments, the chitosan adjuvant includes chitosan in the amount of about 0.1 μg to about 50 mg. In some embodiments, the chitosan adjuvant includes chitosan in the amount of about 0.1 μg to about 25 mg. In some embodiments, the chitosan adjuvant includes chitosan in the amount of about 0.1 μg to about 20 mg. In some embodiments, the chitosan adjuvant includes chitosan in the amount of about 0.1 μg to about 100 mg. In some embodiments, the chitosan adjuvant includes less than 100 mg of chitosan. In some embodiments, the chitosan adjuvant includes less than 90 mg of chitosan. In some embodiments, the chitosan adjuvant includes less than 80 mg of chitosan. In some embodiments, the chitosan adjuvant includes less than 70 mg of chitosan. In some embodiments, the chitosan adjuvant includes less than 60 mg of chitosan. In some embodiments, the chitosan adjuvant includes less than 50 mg of chitosan. In some embodiments, the chitosan adjuvant includes less than 40 mg of chitosan. In some embodiments, the chitosan adjuvant includes less than 30 mg of chitosan. In some embodiments, the chitosan adjuvant includes less than 20 mg of chitosan. In some embodiments, the chitosan adjuvant includes less than 10 mg of chitosan. In some embodiments, the chitosan adjuvant includes less than 5 mg of chitosan. In some embodiments, the chitosan is water-soluble chitosan. In some embodiments, the chitosan is acid-soluble chitosan. In some embodiments, the chitosan is chitosan hydrochloride.

In some embodiments, the chitosan adjuvant may include a buffer. In some embodiments, the buffer may be selected from any pharmaceutically acceptable buffer, including acetic acid, histidine, citrate, Bis-Tris, HEPES, phosphate, MES, and combinations thereof. In some embodiments, the buffer may be present in the amount of 1 mMol to about 100 mMol of the chitosan adjuvant.

In some embodiments, the chitosan adjuvant includes a water-soluble chitosan and a buffer. In some embodiments, the chitosan adjuvant includes chitosan hydrochloride and a buffer. In some embodiments, the chitosan adjuvant includes a buffer and a water-soluble chitosan having a deacetylation of greater than 75% and a viscosity of 1 cp to 200 cp. In some embodiments, the chitosan adjuvant includes a buffer and chitosan hydrochloride having a deacetylation of greater than 75% and a viscosity of 1 cp to 200 cp. In some embodiments, the chitosan adjuvant includes chitosan hydrochloride and a histidine buffer. In some embodiments, the chitosan adjuvant includes a buffer and chitosan hydrochloride having a deacetylation of greater than 75% and a viscosity of 1 cp to 200 cp. In some embodiments, the chitosan adjuvant includes an acetic acid buffer and chitosan hydrochloride having a deacetylation of greater than 85% and a viscosity of 1 cp to 200 cp. In some embodiments, the chitosan adjuvant includes an acetic acid buffer and chitosan hydrochloride having a deacetylation of greater than 75% and a viscosity of 1 cp to 200 cp. In some embodiments, the chitosan adjuvant includes an acetic acid buffer and chitosan hydrochloride having a deacetylation of greater than 85% and a viscosity of 5 cp to 100 cp. In some embodiments, the chitosan adjuvant includes a sodium chloride buffer and chitosan hydrochloride having a deacetylation of greater than 75% and a viscosity of 1 cp to 200 cp. In some embodiments, the chitosan adjuvant includes a sodium chloride buffer and chitosan hydrochloride having a deacetylation of greater than 85% and a viscosity of 5 cp to 100 cp.

In some embodiments, the chitosan adjuvant includes an acid-soluble chitosan and a buffer. In some embodiments, the chitosan adjuvant includes a buffer and an acid-soluble chitosan having a deacetylation of greater than 75% and a viscosity of 1 cp to 200 cp. In some embodiments, the chitosan adjuvant includes an acetic acid buffer and an acid-soluble chitosan having a deacetylation of greater than 75% and a viscosity of 1 cp to 200 cp. In some embodiments, the chitosan adjuvant includes an acetic acid buffer and an acid-soluble chitosan having a deacetylation of greater than 85% and a viscosity of 1 cp to 200 cp. In some embodiments, the chitosan adjuvant includes an acetic acid buffer and an acid-soluble chitosan having a deacetylation of greater than 85% and a viscosity of 5 cp to 100 cp. In some embodiments, the chitosan adjuvant includes an acid-soluble chitosan and a histidine buffer. In some embodiments, the chitosan adjuvant includes a buffer and an acid-soluble chitosan having a deacetylation of greater than 75% and a viscosity of 1 cp to 200 cp. In some embodiments, the chitosan adjuvant includes a sodium chloride buffer and an acid-soluble chitosan having a deacetylation of greater than 75% and a viscosity of 1 cp to 200 cp. In some embodiments, the chitosan adjuvant includes a sodium chloride buffer and an acid-soluble chitosan having a deacetylation of greater than 85% and a viscosity of 5 cp to 100 cp.

In some embodiments, the chitosan adjuvant may include a tonicity modifier. In some embodiments, the tonicity modifier may be selected from any pharmaceutically acceptable tonicity modifiers, such as sodium chloride, potassium chloride, sucrose, trehalose and combinations thereof. In some embodiments the tonicity modifiers may be present in the amount of 10 mM to 500 mM.

In some embodiments, the chitosan adjuvant includes a water-soluble chitosan and a tonicity modifier. In some embodiments, the chitosan adjuvant includes an acid-soluble chitosan and a tonicity modifier. In some embodiments, the chitosan adjuvant includes chitosan hydrochloride and a tonicity modifier. In some embodiments, the chitosan adjuvant includes a tonicity modifier and a water-soluble chitosan having a deacetylation of greater than 75% and a viscosity of 1 cp to 200 cp. In some embodiments, the chitosan adjuvant includes a tonicity modifier and an acid-soluble chitosan having a deacetylation of greater than 75% and a viscosity of 1 cp to 200 cp. In some embodiments, the chitosan adjuvant includes a sodium chloride and water-soluble chitosan having a deacetylation of greater than 85% and a viscosity of 5 cp to 100 cp. In some embodiments, the chitosan adjuvant includes a sodium chloride and an acid-soluble chitosan having a deacetylation of greater than 85% and a viscosity of 5 cp to 100 cp.

In some embodiments, the chitosan adjuvant may include a detergent. In some embodiments, the detergent may be selected from any pharmaceutically acceptable detergents, such as Polysorbate 80, Polysorbate 20, Poloxamer 188, and combinations thereof. In some embodiments the detergents may be present in the amount of 0.001 to 0.2% (w/v).

In some embodiments, the chitosan adjuvant includes a water-soluble chitosan and a detergent. In some embodiments, the chitosan adjuvant includes an acid-soluble chitosan and a detergent. In some embodiments, the chitosan adjuvant includes chitosan hydrochloride and a detergent. In some embodiments, the chitosan adjuvant includes a detergent and a water-soluble chitosan having a deacetylation of greater than 75% and a viscosity of 1 cp to 200 cp. In some embodiments, the chitosan adjuvant includes a detergent and an acid-soluble chitosan having a deacetylation of greater than 75% and a viscosity of 1 cp to 200 cp.

### Preparation of a Water-Soluble Chitosan Adjuvant

In some embodiments, the water-soluble chitosan adjuvant is formed, for example, by first placing approximately 5-5000 mL water in a 10-10000 mL volumetric flask and adding approximately 0.01 to 500 g grams of a buffer, such as histidine and stirring the combination until the solids are dissolved. After the histidine is in solution, approximately 0.001 to 1000 grams of a water-soluble chitosan, such as chitosan hydrochloride may be added to the histidine solution. The combination is then mixed until the water-soluble chitosan solids are dissolved. After the water-soluble chitosan is completely in solution, the flask is then filled to the volumetric line with Q.S. HPLC water to a target volume of 10 to 10000 mL. The pH of the resulting solution is then tested to verify pH. In some embodiments, the preferred pH range of the resulting solution is 5.0-6.5. In some embodiments, the preferred pH range of the resulting solution is 5.3 to 6.2. In some embodiments, the preferred pH range of the resulting solution is 5.5 to 6.0. In some embodiments, the preferred pH range of the resulting solution is 5.6 to 5.9. The water soluble chitosan solution is then sterile filtered in a sterile biosafety cabinet using syringes and a syringe filter system, such as, a Sterile Acrodisc® Syringe Filter with a Supor® Membrane (Pall® Corporation) to sterile filter the solution. Alternatively, an appropriately sized capsule filter with PES membrane can be used for sterile filtration in a biological safety cabinet.

### Preparation of an Acid-Soluble Chitosan Adjuvant

In some embodiments, the acid-soluble chitosan adjuvant is formed, for example, by first placing approximately 5 to 5,000 mL of a buffer, such as 1% acetic acid, in a 10 to 10,000 mL volumetric flask and adding approximately 0.001 to 1,000 grams of an acid-soluble chitosan, and stirring the combination until the chitosan solids are dissolved. After the acid-soluble chitosan is in solution, solid powder of a buffer, such as histidine, will be weighed out to achieve a final solution concentration of 10 to 500 mM may be added to the acid-soluble chitosan solution. The pH of the acid-soluble chitosan solution was adjusted to a preferred range of approximately 5.0-6.5 using either the amount buffer, such as histidine added or using a basic solution such as a 20% w/v sodium hydroxide solution. In some embodiments, the preferred pH range of the resulting solution is 5.3 to 6.2. In some embodiments, the preferred pH range of the resulting solution is 5.5 to 6.0. In some embodiments, the preferred pH range of the resulting solution is 5.6 to 5.9. The acid-soluble chitosan solution is then sterile filtered in a sterile biosafety cabinet using syringes and a syringe filter system, such as, a Sterile Acrodisc® Syringe Filter with a Supor® Membrane (Pall® Corporation) to sterile filter the solution. Alternatively, an appropriately sized capsule filter with PES membranes can be used for sterile filtration in a biological safety cabinet.

### The VLPs

As stated above, the pharmaceutical compositions and formulations of the present invention comprise at least one HPV VLP type, such as HPV 16 or 18. In particular embodiments of the compositions disclosed herein, the vaccine further comprises VLPs of at least one additional HPV type. In further embodiments, the at least one additional HPV type is selected from the group consisting of: 6, 11, 16, 18, 26, 31, 33, 35, 39, 45, 51, 52, 53, 55, 56, 58, 59, 66, 68, 69, 70, 73, and 82. In some embodiments, the at least one additional HPV type includes HPV 16 and 18. In some embodiments, the at least one additional HPV type includes HPV 6, 11, 16, and 18. In some embodiments, the at least one additional HPV type includes HPV 6, 18, 52, and 58. In some embodiments, the at least one additional HPV type includes HPV 6, 11, 16, 18, 31, 45, 52, and 58. In some embodiments, the at least one additional HPV type includes HPV 6, 11, 16, 18, 33, 45, 52, and 58. In some embodiments, the at least one additional HPV type includes HPV 6, 11, 16, 18, 31, 33, 45, 52, and 58. In some embodiments, the at least one additional HPV type includes 6, 11, 16, 18, 31, 33, 45, 52, and 59. In some embodiments, the at least one additional HPV type includes HPV 6, 11, 16, 18, 31, 33, 45, 53, and 58. In some embodiments, the at least one additional HPV type includes HPV 6, 11, 16, 18, 31, 33, 45, 53, and 59. In some embodiments, the at least one additional HPV type includes HPV 6, 11, 16, 18, 31, 33, 35, 45, 52, and 58. In some embodiments, the at least one additional HPV type includes HPV 6, 11, 16, 18, 31, 33, 35, 45, 52, 58, and 59. In some embodiments, the at least one additional HPV type includes HPV 6, 11, 16, 18, 31, 33, 45, 52, 58, 59, and 68. In some embodiments, the at least one additional HPV type includes HPV 6, 11, 16, 18, 31, 33, 35, 39, 45, 51, 52, 56, 58, and 59. In some embodiments, the at least one additional HPV type includes HPV 6, 11, 16, 18, 26, 31, 33, 35, 45, 51, 52, 58, 59, and 69. In some embodiments, the at least one additional HPV type includes HPV 6, 11, 16, 18, 26, 31, 33, 35, 39, 45, 51, 52, 58, 59, 68, 69, and 70. In some embodiments, the at least one additional HPV type includes HPV 6, 11, 16, 18, 26, 31, 33, 35, 39, 45, 51, 52, 53, 56, 58, 59, 66, 68, 69, and 70. The pharmaceutical compositions of the present invention comprise HPV VLPs comprised of recombinant L1 or recombinant L1+L2 proteins of HPV. HPV L1 or L1+L2 protein can be expressed recombinantly by molecular cloning of L1 or L1+L2 DNA into an expression vector containing a suitable promoter and other appropriate transcription regulatory elements, and transferred into prokaryotic or eukaryotic host cells to produce recombinant protein. Techniques for such manipulations are fully described by Sambrook et al. (Molecular Cloning: A Laboratory Manual; Cold Spring Harbor Laboratory, Cold Spring Harbor, N.Y., (1989)), which is hereby incorporated by reference. VLPs can self-assemble when L1 protein is recombinantly expressed in a host cell.

The recombinant HPV L1 proteins of the present invention may be any full-length L1 protein sequence that can be found in nature or any mutated or truncated L1 protein that is capable of self-assembling into VLPs. In particular embodiments of the invention, the pharmaceutical compositions and vaccines described herein comprise HPV VLPs comprised of recombinant HPV L1 protein and do not contain HPV L2 protein. In certain embodiments, the vaccine compositions or pharmaceutical compositions described herein comprise HPV VLPs comprised of a full-length recombinant HPV L1 protein. In other embodiments, the HPV VLPs are comprised of truncated HPV L1 protein, e.g., L1 protein that are truncated at the C-terminal end. L1 protein sequences for use in the present invention can be determined by isolating DNA from one or more clinical samples containing an HPV type of choice, determining the sequence of the HPV L1 DNA sequence, and translating the DNA sequence into an amino acid sequence using the genetic code. Many exemplary L1 sequences suitable for use in the present invention can be found in the literature. See, e.g., U.S. Pat. Nos. 5,820,870; 7,250,170; 7,276,243; 7,482,428; 7,976,848; 7,498,036; 7,700,103; 7,744,892; and U.S. Pat. No. 5,437,951; Kirii et al. (Virology 185(1): 424-427 (1991)). Further L1 proteins that are useful in the compositions and formulations of the present invention include biologically active fragments and/or mutants of an HPV L1 sequence, including but not necessarily limited to amino acid substitutions, deletions, additions, amino terminal truncations and carboxy-terminal truncations, such that these mutations provide for L1 proteins or protein fragments that are capable of forming a VLP. See, e.g., International Publication WO 2006/114312 and U.S. Pat. No. 6,599,508. Appropriate host cells for the expression of recombinant HPV L1 or recombinant L1+L2 and subsequent self-assembly of VLPs include, but are not limited to yeast cells, insect cells, mammalian cells or bacteria. In exemplary embodiments of the invention, the VLPs are produced in yeast cells such as a yeast selected from the group consisting of: Saccharomyces cerevisiae, Hansenula polymorpha, Pichia pastoris, Kluyveromyces fragilis, Kluyveromyces lactis, and Schizosaccharomyces pombe. In particular embodiments, the HPV VLPs are produced in Saccharomyces cerevisiae cells. Expression of HPV VLPs in yeast cells offers the advantages of being cost-effective and easily adapted to large-scale growth in fermenters.

The present invention also includes pharmaceutical compositions comprising mutant forms of HPV VLPs, such as HPV VLPs that comprise biologically active fragments and/or mutants of an HPV L1 or L2 protein, including but not necessarily limited to amino acid substitutions, deletions, additions, amino terminal truncations and carboxy-terminal truncations such that these mutations provide for proteins or protein fragments of therapeutic or prophylactic use and would be useful for HPV VLP vaccine development. Any such mutant form of an HPV L1 protein should be capable of forming VLPs and of provoking an immune response against the desired HPV type when administered to a human.

Additionally, one of skill in the art will recognize that the HPV L1 or L1+L2 proteins, which are used to self-assemble VLPs for inclusion in the compositions disclosed herein, may be encoded by a full-length wild-type HPV L1 or L2 polynucleotide, or may be encoded by a fragment or mutant of the known wild-type sequence. Wild-type polynucleotide sequences that encode mRNA expressing HPV L1 or L2 protein are available in the art. Any mutant polynucleotide will encode either a protein or protein fragment which at least substantially mimics the pharmacological properties of an HPV L1 or L2 protein, including the ability to form VLPs that are able to provoke an immune response against the HPV type of interest when administered to a human. Any such polynucleotide includes but is not necessarily limited to: nucleotide substitutions, deletions, additions, amino-terminal truncations and carboxy-terminal truncations.

The amount of virus-like particles of each HPV type to be included in the formulations and compositions of the present invention will depend on the immunogenicity of the expressed gene product. In general, a therapeutically effective dose of VLPs of any of the at least one HPV type is about 1 μg to about 300 μg. In some embodiments, a therapeutically effective dose of VLPs of any of the at least one HPV type is about 1 μg to 200 μg. In some embodiments, a therapeutically effective dose of VLPs of any of the at least one HPV type is about 1 μg to 100 μg. In some embodiments, a therapeutically effective dose of VLPs of any of the at least one HPV type is about 10 μg to 200 μg. In some embodiments, a therapeutically effective dose of VLPs of any of the at least one HPV type is about 10 μg to 100 μg. In some embodiments, a therapeutically effective dose of VLPs of any of the at least one HPV type is about 10 μg to 80 μg. In some embodiments, a therapeutically effective dose of VLPs of any of the at least one HPV type is about preferably about 20 μg to 60 μg.

In some embodiments, a 0.5 mL dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 45 L1 protein,
10-120 μg of VLPs of HPV Type 52 L1 protein,
10-120 μg of VLPs of HPV Type 58 L1 protein.

In some embodiments, a 0.5 mL dose of a composition or vaccine including VLPs of the at least one HPV type includes:
15-120 μg of VLPs of HPV Type 6 L1 protein,
20-150 μg of VLPs of HPV Type 11 L1 protein,
30-210 μg of VLPs of HPV Type 16 L1 protein,
20-150 μg of VLPs of HPV Type 18 L1 protein,
10-90 μg of VLPs of HPV Type 31 L1 protein,
10-90 μg of VLPs of HPV Type 33 L1 protein,
10-90 μg of VLPs of HPV Type 45 L1 protein,
10-90 μg of VLPs of HPV Type 52 L1 protein,
10-90 μg of VLPs of HPV Type 58 L1 protein.

In some embodiments, a 0.5 mL dose of a composition or vaccine including VLPs of the at least one HPV type includes:
15-80 μg of VLPs of HPV Type 6 L1 protein,
20-100 μg of VLPs of HPV Type 11 L1 protein,
30-140 μg of VLPs of HPV Type 16 L1 protein,
20-100 μg of VLPs of HPV Type 18 L1 protein,
10-60 μg of VLPs of HPV Type 31 L1 protein,
10-60 μg of VLPs of HPV Type 33 L1 protein,
10-60 μg of VLPs of HPV Type 45 L1 protein,
10-60 μg of VLPs of HPV Type 52 L1 protein,
10-60 μg of VLPs of HPV Type 58 L1 protein.

In some embodiments, a 0.5 mL dose of a composition or vaccine including VLPs of the at least one HPV type includes:
15-40 μg of VLPs of HPV Type 6 L1 protein,
20-50 μg of VLPs of HPV Type 11 L1 protein,
30-70 μg of VLPs of HPV Type 16 L1 protein,
20-50 μg of VLPs of HPV Type 18 L1 protein,
10-30 μg of VLPs of HPV Type 31 L1 protein,
10-30 μg of VLPs of HPV Type 33 L1 protein,
10-30 μg of VLPs of HPV Type 45 L1 protein,
10-30 μg of VLPs of HPV Type 52 L1 protein,
10-30 μg of VLPs of HPV Type 58 L1 protein.

In some embodiments, a 0.5 mL dose of a composition or vaccine including VLPs of the at least one HPV type includes:
90 μg of VLPs of HPV Type 6 L1 protein,
120 μg of VLPs of HPV Type 11 L1 protein,
180 μg of VLPs of HPV Type 16 L1 protein,
120 μg of VLPs of HPV Type 18 L1 protein,
60 μg of VLPs of HPV Type 31 L1 protein,
60 μg of VLPs of HPV Type 33 L1 protein,
60 μg of VLPs of HPV Type 45 L1 protein,
60 μg of VLPs of HPV Type 52 L1 protein,
60 μg of VLPs of HPV Type 58 L1 protein.

In some embodiments, a 0.5 mL dose of a composition or vaccine including VLPs of the at least one HPV type includes:
60 μg of VLPs of HPV Type 6 L1 protein,
80 μg of VLPs of HPV Type 11 L1 protein,
120 μg of VLPs of HPV Type 16 L1 protein,
80 μg of VLPs of HPV Type 18 L1 protein,
40 μg of VLPs of HPV Type 31 L1 protein,
40 μg of VLPs of HPV Type 33 L1 protein,
40 μg of VLPs of HPV Type 45 L1 protein,
40 μg of VLPs of HPV Type 52 L1 protein,
40 μg of VLPs of HPV Type 58 L1 protein.

In some embodiments, a 0.5 mL dose of a composition or vaccine including VLPs of the at least one HPV type includes:
30 μg of VLPs of HPV Type 6 L1 protein,
40 μg of VLPs of HPV Type 11 L1 protein,
60 μg of VLPs of HPV Type 16 L1 protein,
40 μg of VLPs of HPV Type 18 L1 protein,
20 μg of VLPs of HPV Type 31 L1 protein,
20 μg of VLPs of HPV Type 33 L1 protein,
20 μg of VLPs of HPV Type 45 L1 protein,
20 μg of VLPs of HPV Type 52 L1 protein,
20 μg of VLPs of HPV Type 58 L1 protein.

### The Aluminum Adjuvant

The aluminum adjuvant of the present invention may be in the form of aluminum hydroxide (Al(OH)₃), aluminum phosphate (AlPO₄), aluminum hydroxyphosphate, amorphous aluminum hydroxyphosphate sulfate (AAHS) or so-called “alum” (KAl(SO₄)-12H₂O) (see Klein et al., Analysis of aluminum hydroxyphosphate vaccine adjuvants by (27)A1 MAS NMR., J Pharm. Sci. 89(3): 311-21 (2000)). In exemplary embodiments of the invention provided herein, the aluminum adjuvant is aluminum hydroxyphosphate or AAHS. The ratio of phosphate to aluminum in the aluminum adjuvant can range from 0 to 1.3. In preferred embodiments of this aspect of the invention, the phosphate to aluminum ratio is within the range of 0.1 to 0.70. In particularly preferred embodiments, the phosphate to aluminum ratio is within the range of 0.2 to 0.50.

In some embodiments of the invention, the aluminum adjuvant is in the form of AAHS. AAHS carries zero charge at neutral pH, while Al(OH)₃ carries a net positive charge and AlPO₄ typically carries a net negative charge at neutral pH. AAHS has a higher capacity to bind HPV VLPs than Al(OH)₃. In addition, VLPs adsorbed to AAHS can induce a greater humoral immune response in mice than VLPs adsorbed to Al(OH)₃. Caulfield et al., Human Vaccines 3: 139-146 (2007). While not wishing to be bound by theory, it is possible that net charge of the aluminum adjuvant can affect its ability to bind the VLP antigen, with strongly charged adjuvants unable to bind antigen as strongly as neutral charged adjuvants. For this reason, it is preferred that the aluminum adjuvant of the pharmaceutical compositions of the present invention have zero point surface charge at neutral pH. One of skill in the art will be able to vary the buffer, salt concentration and/or percent of free phosphate in order to allow a zero point surface charge at neutral pH.

One of skill in the art will be able to determine an optimal dosage of aluminum adjuvant that is both safe and effective at increasing the immune response to the targeted HPV type(s). For a discussion of the safety profile of aluminum, as well as amounts of aluminum included in FDA-licensed vaccines, see Baylor et al., Vaccine 20: S18-S23 (2002). In some embodiments, the aluminum adjuvant may be present in the amount of about 100 to 3600 μg/dose (200 to 7200 μg/mL concentration). In some embodiments, the aluminum adjuvant may be present in the amount of about 100 to 2700 μg/dose (200 to 5400 μg/mL concentration). In some embodiments, the aluminum adjuvant may be present in the amount of about 100 to 1800 μg/dose (200 to 3600 μg/mL concentration). In some embodiments, the aluminum adjuvant may be present in the amount of about 100 to 900 μg/dose (200 to 1800 μg/mL concentration). In some embodiments of the formulations and compositions of the present invention, there is between 200 and 300 μg aluminum adjuvant per dose of vaccine. In alternative embodiments of the formulations and compositions of the present invention, there is between 300 and 500 μg aluminum adjuvant per dose of vaccine. In alternative embodiments of the formulations and compositions of the present invention, there is between 400 and 1200 μg aluminum adjuvant per dose of vaccine. In alternative embodiments of the formulations and compositions of the present invention, there is between 1200 and 2000 μg aluminum adjuvant per dose of vaccine. In some embodiments of the formulations and compositions of the present invention, there is less than 2000 μg aluminum adjuvant per dose of vaccine. In some embodiments of the formulations and compositions of the present invention, there is less than 1500 μg aluminum adjuvant per dose of vaccine. In some embodiments of the formulations and compositions of the present invention, there is less than 1000 μg aluminum adjuvant per dose of vaccine. In some embodiments of the formulations and compositions of the present invention, there is less than 500 μg aluminum adjuvant per dose of vaccine. In some embodiments of the formulations and compositions of the present invention, there is less than 400 μg aluminum adjuvant per dose of vaccine. In some embodiments of the formulations and compositions of the present invention, there is less than 300 μg aluminum adjuvant per dose of vaccine. In some embodiments of the formulations and compositions of the present invention, there is less than 200 μg aluminum adjuvant per dose of vaccine. In some embodiments of the formulations and compositions of the present invention, there is less than 100 μg aluminum adjuvant per dose of vaccine.

### The HPV VLP-Based Vaccine

Any HPV VLP-based vaccine is suitable for use in the pharmaceutical compositions and methods of the present invention. Known HPV VLP vaccines can be modified to include both an aluminum adjuvant and a chitosan adjuvant. New vaccines can be developed according to the invention described herein that comprise at least one HPV type, optionally in the form of an HPV VLP adsorbed to an aluminum adjuvant, in combination with a chitosan adjuvant. Additionally, new vaccines can be developed according to the invention described herein that comprise at least one HPV type in the form of an HPV VLP adsorbed to an aluminum adjuvant in combination with a chitosan adjuvant.

One exemplary HPV vaccine is a bivalent vaccine protective against HPV 16 and 18, which is known commercially as CERVARIX® (GlaxoSmithKline Biologicals, Rixensart, Belgium). Another exemplary HPV VLP vaccine is a non-infectious recombinant, quadrivalent vaccine prepared from highly purified VLPs of the major capsid (L1) protein of HPV types 6, 11, 16, and 18, and may be referred to herein by its proprietary name GARDASIL® (Merck & Co., Inc., Kenilworth, N.J., USA), see Bryan, J. T. Vaccine 25(16): 3001-6 (2007); Shi et al. Clinical Pharmacology and Therapeutics 81(2): 259-64 (2007). Another exemplary HPV VLP vaccine is the nine-valent vaccine marketed for prevention of HPV (that includes the capsid (L1) protein of HPV types 6, 11, 16, 18, 31, 33, 45, 52, and 58), which is referred to herein by its proprietary name GARDASIL®9 (Merck & Co., Inc., Kenilworth, N.J., USA).

In some embodiments, the vaccine dose includes, in addition to VLPs, an aluminum adjuvant (as amorphous aluminum hydroxyphosphate sulfate), sodium chloride, L-histidine, polysorbate 80, sodium borate, and water. In some embodiments, the HPV vaccine may include 100-3500 μg aluminum adjuvant, 1-50 mg sodium chloride, 0.05-10 mg L-histidine, 1-100 μg polysorbate, 1-100 μg sodium borate, and water. In some embodiments, the HPV vaccine may include about 500 μg aluminum adjuvant, about 9.56 mg sodium chloride, about 0.78 mg L-histidine, about 50 μg polysorbate 80, about 35 μg sodium borate, and water for injection. Known HPV VLP vaccines can be modified to include both an aluminum adjuvant and a chitosan adjuvant in accordance with the present invention.

In some embodiments of the invention, the pharmaceutical compositions and formulations comprise HPV VLP-based vaccines, or HPV VLPs as described herein, that are monovalent, bivalent, trivalent, quadrivalent, 5-valent, 6-valent, 7-valent, 8-valent or 9-valent. In particular embodiments, the pharmaceutical compositions and formulations are 9-valent. In some embodiments, the pharmaceutical compositions comprise HPV VLP-based vaccines, or HPV VLPs as described herein, with more than four different types of HPV VLPs. For example, the pharmaceutical compositions and formulations of the present invention may include HPV VLP-based vaccines, or HPV VLPs as described herein, that are 8-valent, 9-valent, 10-valent, and so forth. For example, pharmaceutical compositions comprising VLPs of HPV 16 and/or HPV 18, without the inclusion of other HPV VLP types, are included within the scope of the invention. Multi-valent vaccines comprising different HPV VLPs than the HPV types included in GARDASIL® or GARDASIL®9 are also contemplated herein.

In some embodiments, the VLPs of HPV types 6 and 11 are included. In some embodiments, the VLPs of HPV types 16, 31, and 35 are included. In some embodiments, the VLPs of HPV types 18, 45, and 59 are included. In some embodiments, the VLPs of HPV types 26, 51, and 69 are included. In some embodiments, the VLPs of HPV types 33, 52, and 58 are included. In some embodiments, the VLPs of HPV types 39, 68, and 70 are included. In some embodiments, the VLPs of HPV types 53, 56, and 66 are included.

In some embodiments, the VLPs of HPV types 16 and 18 are included. In some embodiments, the VLPs of HPV types 6, 11, 16, and 18 are included. In some embodiments, the VLPs of HPV types 6, 18, 52, and 58 are included. In some embodiments, the VLPs of HPV types 6, 11, 16, 18, 31, 45, 52, and 58 are included. In some embodiments, the VLPs of HPV types 6, 11, 16, 18, 33, 45, 52, and 58 are included. In some embodiments, the VLPs of HPV types 6, 11, 16, 18, 31, 33, 45, 52, and 58 are included. In some embodiments, the VLPs of HPV types 6, 11, 16, 18, 31, 33, 45, 52, and 59 are included. In some embodiments, the VLPs of HPV types 6, 11, 16, 18, 31, 33, 45, 53, and 58 are included. In some embodiments, the VLPs of HPV types 6, 11, 16, 18, 31, 33, 45, 53, and 59 are included. In some embodiments, the VLPs of HPV types 6, 11, 16, 18, 31, 33, 35, 45, 52, and 58 are included. In some embodiments, the VLPs of HPV types 6, 11, 16, 18, 31, 33, 35, 45, 52, 58, and 59 are included. In some embodiments, the VLPs of HPV types 6, 11, 16, 18, 31, 33, 45, 52, 58, 59, and 68 are included. In some embodiments, the VLPs of HPV types 6, 11, 16, 18, 31, 33, 35, 39, 45, 51, 52, 56, 58, and 59 are included. In some embodiments, the VLPs of HPV types 6, 11, 16, 18, 26, 31, 33, 35, 45, 51, 52, 58, 59, and 69 are included. In some embodiments, the VLPs of HPV types 6, 11, 16, 18, 26, 31, 33, 35, 39, 45, 51, 52, 58, 59, 68, 69, and 70 are included. In some embodiments, the VLPs of HPV types 6, 11, 16, 18, 26, 31, 33, 35, 39, 45, 51, 52, 53, 56, 58, 59, 66, 68, 69, and 70 are included.

In some embodiments, the pharmaceutical compositions and formulations comprise HPV VLP-based vaccines and/or antigens as listed in Table I below:

TABLE I

| Name | Antigen | Adjuvant | Party |
|---|---|---|---|
| CERVARIX ® | L1 VLP of HPV-16 and HPV- | Aluminum | GlaxoSmithKline |
| (2vHPV vaccine) | 18 | hydroxide and | Biologics (Rixensart, |
| | | MPL | Belgium) |
| GARDASIL ® | L1 VLP of HPV-6, HPV-11, | AHSS | Merck & Co., Inc., |
| (4vHPV vaccine) | HPV-16 and HPV-18 | | Kenilworth NJ USA |
| GARDASIL ® 9 | L1 VLP of HPV-6, HPV-11, | AHSS | Merck& Co., Inc., |
| (9vHPV vaccine) | HPV-16, HPV-18, HPV-31, | | Kenilworth NJ USA |
| | HPV-33, HPV-45, HPV-52 | | |
| | and HPV-58 | | |
| CECOLIN ® | L1 VLP of HPV-16 and HPV- | Aluminum | Xiamen Innovax |
| | 18 | hydroxide | |
| GEOCOLIN ® | L1 VLP of HPV-6 and HPV- | Aluminum | Xiamen Innovax |
| | 11 | hydroxide | |
| L1 capsomers | L1 capsomers of HPV-16 | unknown | R. Garcea, University |
| | | | of Colorado- Boulder |
| RG1-VLP | HPV-16 L1-L2 (17-36) VLP | Aluminum | R. Kimbauer, NCI, |
| | | hydroxide | Pathovax LLC |
| L2-AAV | L2 peptides of HPV-16 and | unknown | 2A Pharma |
| | HPV-31 displayed on AAV | | |
| | VLP | | |
| L2 multimer | Fusion protein of L2 ~11-88 | Alum | Sanofi, BravoVax |
| | of HPV-6, HPV-16, HPV-18, | | |
| | HPV-31 and HPV-39 | | |
| L2-thioredoxin | L2 peptide displayed on | unknown | M. Muller, DKFZ |
| | thioredoxin | | |
| AX03 | L2 peptide displayed on | unknown | Agilvax, NIAID |
| | bacteriophage | | |
| L1-E7 VLP | HPV-16 L1-E7 VLP | None | Medigene AG |
| TA-CIN | HPV-16 L2E7E6 fusion | None | Cantab |
| | protein | | Pharmaceuticals, |
| | | | Xenova |
| TA-GW | HPV-6 L2E7 fusion protein | Aluminum | Cantab |
| | | hydroxide or | Pharmaceuticals, GSK |
| | | AS03 | |

### Single Dose Vaccine Compositions

In some embodiments, a single-dose vaccine composition is provided that is a pharmaceutical composition (i.e., includes a pharmaceutically acceptable carrier) and includes a chitosan adjuvant and HPV VLP particles of at least one HPV type. In some embodiments, a single-dose vaccine composition is provided that includes a chitosan adjuvant and HPV VLP particles of at least two HPV types. In some embodiments, a single-dose vaccine composition is provided that includes a chitosan adjuvant and HPV VLP particles of at least four HPV types. In some embodiments, a vaccine composition is provided that includes a chitosan adjuvant and HPV VLP particles of at least nine HPV types.

In some embodiments, a single-dose vaccine composition is provided that includes a chitosan adjuvant and HPV VLP particles of at least one HPV type and an aluminum adjuvant. In some embodiments, a single-dose vaccine composition is provided that includes a chitosan adjuvant and HPV VLP particles of at least two HPV types and an aluminum adjuvant. In some embodiments, a single-dose vaccine composition is provided that includes a chitosan adjuvant and HPV VLP particles of at least four HPV types and an aluminum adjuvant. In some embodiments, a vaccine composition is provided that includes a chitosan adjuvant and HPV VLP particles of at least nine HPV types and an aluminum adjuvant.

In some embodiments, a single-dose vaccine composition is provided that includes (a) about 0.1 μg to about 50 mg chitosan, and (b) HPV VLP particles of at least one HPV type, wherein each of the HPV VLPs, when present in the single dose vaccine composition, are present in a concentration of about 1 μg to about 300 μg per 0.5 mL of the single-dose vaccine composition and wherein the total VLP concentration is between about 10 μg to about 2000 μg per 0.5 mL of the single-dose vaccine composition. In some embodiments, a single-dose vaccine composition is provided that includes (a) about 0.1 μg to about 50 mg chitosan, (b) about 100 μg to about 3500 μg aluminum adjuvant, and (c) HPV VLP particles of at least one HPV type, wherein each of the HPV VLPs, when present in the single dose vaccine composition, are present in a concentration of about 1 μg to about 180 μg per 0.5 mL of the single-dose vaccine composition and wherein the total VLP concentration is between about 10 μg to about 2000 μg per 0.5 mL of the single-dose vaccine composition.

In some embodiments, a single-dose vaccine composition is provided that includes (a) about 0.1 μg to about 50 mg chitosan, about 1 μg to about 2000 μg HPV VLP particles of at least two HPV types, and about 100 μg to about 2700 μs aluminum adjuvant. In some embodiments, a single-dose vaccine composition is provided that includes (a) about 0.1 μg to about 50 mg chitosan, HPV VLP particles of at least four HPV types, and about 100 μg to about 3500 μg aluminum adjuvant.

In some embodiments, a single-dose vaccine composition is provided that includes 0.1 to about 50 mg chitosan, and 1 μg to about 100 μg of each HPV VLP present in the single dose vaccine composition. In some embodiments, a single-dose vaccine composition is provided that includes 0.1 μg to about 50 mg chitosan and 2 μg to about 600 μg of HPV VLPs of two HPV types (i.e., the single-dose vaccine is a bivalent VLP HPV vaccine). In some embodiments, a single-dose vaccine composition is provided that includes 0.1 μg to about 50 mg chitosan and 4 μg to about 1200 μg of HPV VLPs of four HPV types (i.e., the single-dose vaccine is a quadrivalent VLP HPV vaccine). In some embodiments, a single dose vaccine composition is provided that includes 0.1 μg to about 50 mg chitosan and 9 μg to about 2700 μg of HPV VLPs of nine (9) HPV types (i.e., the single-dose vaccine is 9-valent VLP HPV vaccine). In some embodiments, a single dose vaccine composition is provided that includes 0.1 μg to about 50 mg chitosan and 20 μg to about 6000 μg of HPV VLPs of twenty (20) HPV types (i.e., the single-dose vaccine is a 20-valent VLP HPV vaccine). In some embodiments, the single-dose vaccine composition also includes about 100 μg to about 2700 μg aluminum adjuvant.

In some embodiments, a single-dose vaccine composition is provided that includes 0.1 to about 50 mg chitosan, 1 μg to about 300 μg of a monovalent VLP HPV, and (c) 100 μg to about 2700 μg aluminum adjuvant. In some embodiments, a single-dose vaccine composition is provided that includes 0.1 μg to about 50 mg chitosan, 1 μg to about 300 per VLP, of a bivalent VLP HPV (i.e., HPV VLPs of two HPV types), and 100 μg to about 3500 μg aluminum adjuvant. In some embodiments, a single-dose vaccine composition is provided that includes (a) 0.1 μg to about 50 mg chitosan, (b) 1 μg to about 300 μg, per VLP, of a quadrivalent VLP HPV (i.e., HPV VLPs of four HPV types), and (c) 100 μg to about 3500 μg aluminum adjuvant. In some embodiments, a single-dose vaccine composition is provided that (a) includes 0.1 μg to about 50 mg chitosan, (b) 1 μg to about 300 μg, per VLP, of a 9-valent VLP HPV (i.e., HPV VLPs of 9 HPV types), and (c) 100 μg to about 3500 μg aluminum adjuvant. In some embodiments, a single-dose vaccine composition is provided that includes (a) includes 0.1 μg to about 50 mg chitosan, (b) 1 μg to about 300 μg, per VLP, of a 20-valent VLP HPV (i.e., HPV VLPS of 20 HPV types), and (c) 100 μg to about 3500 μg aluminum adjuvant.

In some embodiments, the single-dose vaccine composition includes (a) 1 μg to about 300 μg, per VLP, of HPV VLPs (HPV types 16 and 18) and (b) 0.1 μg to about 50 mg chitosan. In some embodiments, the single-dose vaccine composition includes (a) 1 μg to about 300 μg, per VLP, of HPV VLPs (HPV types 6, 11, 16, and 18) and (b) 0.1 μg to about 50 mg chitosan. In some embodiments, the single-dose vaccine composition includes (a) 1 μg to about 300 μg, per VLP, of HPV VLPs (HPV types 6, 11, 16, 18, 31, 33, 45, 52, and 58) and (b) 0.1 μg to about 50 mg chitosan.

In some embodiments, the single-dose vaccine composition includes 1 μg to about 300 μg, per VLP, of HPV VLPs (HPV types 16 and 18), 100 μg to about 3500 μg of an aluminum adjuvant, and 0.1 μg to about 50 mg chitosan. In some embodiments, the single-dose vaccine composition includes 1 μg to about 300 μg, per VLP, of HPV VLPs (HPV types 6, 11, 16, and 18), 100 μg to about 3500 μg of an aluminum adjuvant, and 0.1 μg to about 50 mg chitosan. In some embodiments, the single-dose vaccine composition includes 1 μg to about 300 μg, per VLP, of HPV VLPs (HPV types 6, 11, 16, 18, 31, 33, 45, 52, and 58), 100 μg to about 3500 μg of an aluminum adjuvant, and 0.1 μg to about 50 mg chitosan.

The vaccines of the invention comprise VLPs containing the antigenic determinants required to induce the generation of neutralizing antibodies in the subject. The vaccines are expected to be sufficiently safe to be administered without the risk of clinical infection, have no toxic side effects, are stable, compatible with conventional carriers and can be administered effectively. In some embodiments, a chitosan adjuvant of the present invention may be combined with a Human Papillomavirus Bivalent (Types 16 and 18) Vaccine, Recombinant. In some embodiments, the chitosan adjuvant of the present invention may be combined with CERVARIX®. In some embodiments, a chitosan adjuvant of the present invention may be combined with a Human Papillomavirus Quadrivalent (Types 6, 11, 16, 18) Vaccine, Recombinant. In some embodiments, a chitosan adjuvant of the present invention may be combined with GARDASIL®. In some embodiments, a chitosan adjuvant of the present invention may be combined with a Human Papillomavirus 9-valent Vaccine, Recombinant. In some embodiments, a chitosan adjuvant of the present invention may be combined with GARDASIL® 9.

Pharmaceutical compositions, formulations, and single-dose vaccines of the present invention may be administered subcutaneously, topically, orally, on the mucosa, intravenously, or intramuscularly. The pharmaceutical compositions, formulations, and vaccines are administered in an amount sufficient to elicit a protective response. Vaccines, pharmaceutical compositions and formulations can be administered by various routes, for example, orally, parenterally, subcutaneously, on the mucosa, or intramuscularly. The dose administered may vary depending on the general condition, sex, weight and age of the patient, the route of administration and the type of HPV VLP in the vaccine. The vaccine, pharmaceutical composition, for formulation may be in the form of a capsule, suspension, elixir or solution. It may be formulated with an immunologically acceptable carrier.

### Kits of the Invention

Also provided herein are kits including any of the pharmaceutical compositions of single dose vaccines as described above and instructions for use.

Also provided herein are kits including (a) a pharmaceutical composition comprising HPV VLPs of at least one type of HPV and (b) a chitosan adjuvant.

In some embodiments of the kits, the pharmaceutical composition of (a) comprises HPV VLPs of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiment, the pharmaceutical composition of (a) is an HPV vaccine. In some embodiments, the HPV vaccine is a Human Papillomavirus Bivalent (Types 16 and 18) Vaccine, Recombinant. In some embodiments, the HPV vaccine is CERVARIX®. In some embodiments, the HPV vaccine is a Human Papillomavirus Quadrivalent (Types 6, 11, 16, 18) Vaccine, Recombinant. In some embodiments, the HPV vaccine is GARDASIL®. In some embodiments, the HPV vaccine is a Papillomavirus 9-valent Vaccine, Recombinant. In some embodiments, the HPV vaccine is GARDASIL® 9.

In some embodiments of the kits, the chitosan adjuvant is any of the chitosan adjuvants described herein above. In some embodiments, the kit includes 0.1 μg to 100 mg of a chitosan. In some embodiments, the kit includes 0.1 μg to 100 mg of a water-soluble chitosan. In some embodiments, the kit includes 0.1 μg to 100 mg of an acid-soluble chitosan. In some embodiments, the kit includes 0.1 μg to 100 mg of chitosan hydrochloride. In some embodiments, the kit includes a buffer. In some embodiments, the kit includes a tonicity modifier. In some embodiments, the kit includes a detergent.

In some embodiments of the kits, the kit includes a label or packaging insert that includes a description of the components and/or instructions for use in vivo of the components therein. In some embodiments, the kits include instructions for co-administering (or vaccinating) (a) the pharmaceutical composition or HPV Vaccine and (b) the chitosan adjuvant. In some embodiments, the kits include instructions for admixing (a) the pharmaceutical composition or HPV vaccine and (b) the chitosan adjuvant and subsequentially administering (or vaccinating) the admixture to a patient.

### Methods of Treatment of the Invention

Also provided herein is a method of inducing an immune response to a human papillomavirus (HPV) in a human patient comprising administering to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of inducing an immune response to a human papillomavirus (HPV) in a human patient including administering a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiments, the chitosan adjuvant is formulated separately from the VLPs. In some embodiments, the chitosan adjuvant is formulated with the VLPs. In some embodiments, the chitosan adjuvant and VLPs are field-mixed to form a pharmaceutical composition prior to administration to the patient. In some embodiments, the chitosan adjuvant and VLPs are administered sequentially to a patient.

Also provided herein is a method of inducing an immune response to a human papillomavirus (HPV) in a human patient including co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan adjuvant.

Also provided herein is a method of preventing infection of a human patient by a human papillomavirus (HPV) including administration to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of delivering a pharmaceutical composition to a subject that induces a neutralizing titer against an HPV antigen in the subject that includes administering to the subject a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, whereby the administration of the pharmaceutical composition induces a neutralizing titer against the HPV antigen in the subject, and wherein a single dose of the pharmaceutical composition provides enhanced or comparable neutralizing titers when compared to multiple doses of the same pharmaceutical composition when the same composition is formulated without a chitosan adjuvant.

Also provided herein is a method for preventing cancer of a human patient cancers caused by human papillomavirus (HPV) Types 16, 18, 31, 33, 45, 52, and 58 including administration to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, anal, oropharyngeal, and other head and neck cancers.

Also provided herein is a method for preventing cancer of a human patient caused by HPV Types 6, 11, 16, 18, 31, 33, 45, 52, and 58 including administration to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, and anal precancerous or dysplastic lesions.

Also provided herein is a method for preventing cancer of a human patient caused by HPV Types 6 and 11 including administration to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is genital warts or condyloma acuminata.

Also provided herein is a method for preventing precancerous or dysplastic lesions of a human patient caused by HPV Types 6, 11, 16, 18, 26, 31, 33, 35, 39, 45, 51, 52, 53, 55, 56, 58, 59, 66, 68, 73, and 82 including administration to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the lesions are selected from Cervical intraepithelial neoplasia (CIN) grade 2/3, cervical adenocarcinoma in situ (AIS), Cervical intraepithelial neoplasia (CIN) grade 1, Vulvar intraepithelial neoplasia (VIN) grade 2 and grade 3, Vaginal intraepithelial neoplasia (VaIN) grade 2 and grade 3, Anal intraepithelial neoplasia (AIN) grades 1, 2, and 3. (1.1).

Also provided herein is a method for preventing HPV-related anogenital disease of a human patient caused by HPV Types selected from the group consisting of HPV types: 6, 11, 16, 18, 26, 31, 33, 35, 39, 45, 51, 52, 53, 55, 56, 58, 59, 66, 68, 73, and 82 including administration to the patient a pharmaceutical composition including a chitosan adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Embodiments of the invention also include one or more of the pharmaceutical compositions described herein (i) for use in, (ii) for use as a medicament or composition for, or (iii) for use in the preparation of a medicament for: (a) therapy (e.g., of the human body); (b) medicine; (c) induction of an immune response against HPV types included in the vaccine (d) decreasing the likelihood of HPV infection in a patient; (e) prevention of infection with HPV types in the vaccine, (f) prevention or reduction of the likelihood of cervical cancer, (g) prevention or reduction of the likelihood of vulvar cancer, (h) prevention or reduction of the likelihood of vaginal cancer, (i) prevention or reduction of the likelihood of anal cancer, (j) prevention or reduction of the likelihood of oropharyngeal cancer, (k) prevention or reduction of the likelihood of other head and neck cancers, (k) prevention or reduction of the likelihood of precancerous or dysplastic anal lesions, (1) prevention or reduction of the likelihood of genital warts or condyloma acuminata, (m) prevention or reduction of the likelihood of Cervical intraepithelial neoplasia (CIN) grade 2/3 lesions, (n) prevention or reduction of the likelihood of cervical adenocarcinoma in situ (AIS) lesions, (o) prevention or reduction of the likelihood of Cervical intraepithelial neoplasia (CIN) grade 1 lesions, (p) prevention or reduction of the likelihood of Vulvar intraepithelial neoplasia (VIN) grade 2 and grade 3 lesions, (q) prevention or reduction of the likelihood of Vaginal intraepithelial neoplasia (VaIN) grade 2 and grade 3 lesions, (r) prevention or reduction of the likelihood of Anal intraepithelial neoplasia (AIN) grades 1, 2, and 3 lesions.

In embodiment 1, a pharmaceutical composition is provided that includes virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82, a chitosan; and a pharmaceutically acceptable carrier.

In embodiment 2, the pharmaceutical composition of embodiment 1 is provided, wherein the pharmaceutical composition comprises VLPs of at least HPV types 16 and 18.

In embodiment 3, the pharmaceutical composition of embodiments 1-2 is provided, wherein the pharmaceutical composition comprises VLPs of at least HPV types 6, 11, 16, and 18.

In embodiment 4, the pharmaceutical composition of embodiments 1-3 is provided, wherein the pharmaceutical composition comprises VLPs of at least HPV types 31, 45, 52, and 58.

In embodiment 5, the pharmaceutical composition of any of embodiments 1-4 is provided, wherein the pharmaceutical composition comprises VLPs of at least HPV types 6, 11, 16, 18, 31, 33, 45, 52, and 58.

In embodiment 6, the pharmaceutical composition of any of embodiments 1-5 is provided, wherein the composition comprises VLPs of HPV types 6, 11, 16, 18, 31, 33, 35, 45, 52, and 58.

In embodiment 7, the pharmaceutical composition of any of embodiments 1-6 is provided, wherein the composition comprises VLPs of HPV types 6, 11, 16, 18, 31, 33, 35, 45, 52, 58, and 59.

In embodiment 8, the pharmaceutical composition of any of embodiments 1-5 is provided, wherein the composition comprises VLPs of HPV types 6, 11, 16, 18, 31, 33, 45, 52, 58, 59, and 68.

In embodiment 9, the pharmaceutical composition of any of embodiments 1-7 is provided, wherein the composition comprises VLPs of HPV types 6, 11, 16, 18, 31, 33, 35, 39, 45, 51, 52, 56, 58, and 59.

In embodiment 10, the pharmaceutical composition of any of embodiments 1-6 is provided, wherein the composition comprises VLPs of HPV types 6, 11, 16, 18, 26, 31, 33, 35, 45, 51, 52, 58, 59, and 69.

In embodiment 11, the pharmaceutical composition of any of embodiments 1-7 is provided, wherein the composition comprises VLPs of HPV types 6, 11, 16, 18, 26, 31, 33, 35, 39, 45, 51, 52, 58, 59, 68, 69, and 70.

In embodiment 12, the pharmaceutical composition of any of embodiments 1-11 is provided, wherein the composition comprises VLPs of HPV types 6, 11, 16, 18, 26, 31, 33, 35, 39, 45, 51, 52, 53, 56, 58, 59, 66, 68, 69, and 70.

In embodiment 13, the pharmaceutical composition of any of embodiment 1-5 is provided wherein the pharmaceutical composition further comprises a buffer.

In embodiment 14, the pharmaceutical composition of any of embodiment 1-6 is provided wherein the pharmaceutical composition further comprises aluminum.

In embodiment 15, the pharmaceutical composition of any of embodiment 1-7 is provided wherein the pharmaceutical composition further comprises a salt.

In embodiment 16, the pharmaceutical composition of any of embodiment 1-8 is provided wherein the chitosan is a water soluble chitosan.

In embodiment 17, the pharmaceutical composition of any of embodiment 1-8 is provided wherein the chitosan is an acid soluble chitosan.

In embodiment 18, the pharmaceutical composition of any of claims 1 to 10, is provided wherein the composition is made by mixing an HPV vaccine and a chitosan adjuvant; wherein the HPV vaccine comprises HPV VLPs and a pharmaceutically acceptable carrier and the chitosan adjuvant comprises a chitosan and a pharmaceutically acceptable carrier.

In embodiment 19, the pharmaceutical composition of any of embodiments 1-17 is provided, wherein the composition is made by mixing an HPV vaccine and chitosan.

In embodiment 20, the pharmaceutical composition of any of embodiments 1-16 and 18-19 is provided, wherein the chitosan adjuvant comprises a water-soluble chitosan having a deacetylation of 75% or greater.

In embodiment 21, the pharmaceutical composition of any of embodiments 1-16 and 18-20 is provided, wherein the chitosan adjuvant comprises a water-soluble chitosan having a viscosity in the range of about 1 cP to about 200 cP when measured with a viscosimeter at 20° C. at a standard concentration.

In embodiment 22, the pharmaceutical composition of any of embodiments 1-16 and 18-21 is provided, wherein the chitosan adjuvant comprises a water-soluble chitosan having a deacetylation of 85%-99%.

In embodiment 23, the pharmaceutical composition of any of embodiments 1-16 and 18-22 is provided, wherein the chitosan adjuvant comprises a water-soluble chitosan having a viscosity in the range of about 5 cP to about 10 cP when measured with a viscosimeter at 20° C. at a standard concentration.

In embodiment 24, the pharmaceutical composition of any of embodiments 1-16 and 18-23 is provided, wherein the chitosan adjuvant comprises a water-soluble chitosan having a deacetylation greater than 90% and a viscosity in the range of about 5 cP to about 10 cP when measured with a viscosimeter at 20° C. at a standard concentration.

In embodiment 25, the pharmaceutical composition of any of embodiments 1-16 and 18-24 is provided, wherein the chitosan adjuvant comprises a water-soluble chitosan having a deacetylation greater than 95% and a viscosity of less than 5 cP when measured with a viscosimeter at 20° C. at a standard concentration.

In embodiment 26, the pharmaceutical composition of any of embodiments 1-15 and 17 is provided, wherein the chitosan adjuvant comprises an acid-soluble chitosan having a deacetylation of 75% or greater.

In embodiment 27, the pharmaceutical composition of any of embodiments 1-15, 17, and 26 is provided, wherein the chitosan adjuvant comprises an acid-soluble chitosan having a viscosity in the range of about 1 cP to about 200 cP when measured with a viscosimeter at 20° C. at a standard concentration.

In embodiment 28 the pharmaceutical composition of any of embodiments 1-15, 17, and 26-27 is provided, wherein the chitosan adjuvant comprises an acid-soluble chitosan having a deacetylation of 85%-99%.

In embodiment 29, the pharmaceutical composition of any of embodiments 1-15, 17, and 26-28 is provided, wherein the chitosan adjuvant comprises an acid-soluble chitosan having a viscosity in the range of about 5 cP to about 10 cP when measured with a viscosimeter at 20° C. at a standard concentration.

In embodiment 30, the pharmaceutical composition of any of embodiments 1-15, 17, and 26-28 is provided, wherein the chitosan adjuvant comprises an acid-soluble chitosan having a deacetylation greater than 90% and a viscosity in the range of about 5 cP to about 10 cP when measured with a viscosimeter at 20° C. at a standard concentration.

In embodiment 31, the pharmaceutical composition of any of embodiments 1-15, 17, and 26-28 is provided, wherein the chitosan adjuvant comprises an acid-soluble chitosan having a deacetylation greater than 95% and a viscosity of less than 5 cP when measured with a viscosimeter at 20° C. at a standard concentration.

In embodiment 32, the pharmaceutical composition of any of embodiments 1-31 is provided, wherein the chitosan adjuvant further comprises a buffer.

In embodiment 33, the pharmaceutical composition of embodiment 32 is provided, wherein the buffer is selected from the group consisting of: acetic acid, histidine, citrate, Bis-Tris, HEPES, phosphate, MES, sodium chloride, and combinations thereof.

In embodiment 34, the pharmaceutical composition of any of embodiments 32 and 33 is provided, wherein the buffer is present in the amount of about 1 mMol to about 100 mMol.

In embodiment 35, the pharmaceutical composition of any of embodiments 1-34 is provided, wherein the chitosan adjuvant further comprises a tonicity modifier.

In embodiment 36, the pharmaceutical composition of embodiment 35 is provided, wherein the tonicity modifier is selected from the group consisting of: sodium chloride, potassium chloride, sucrose, trehalose and combinations thereof.

In embodiment 37, the pharmaceutical composition of any of embodiments 35 and 36 is provided, wherein the tonicity modifier is present in the amount of about 10 mMol to about 500 mMol.

In embodiment 38, the pharmaceutical composition of any of embodiments 1-37 is provided, wherein the chitosan adjuvant further comprises a detergent.

In embodiment 39, the pharmaceutical composition of embodiment 38 is provided, wherein the detergent is selected from the group consisting of Polysorbate 80, Polysorbate 20, Poloxamer 188, and combinations thereof.

In embodiment 40, the pharmaceutical composition of any of embodiments 38 and 39 is provided, wherein the detergent is present in the amount of about 0.001% (w/v) to about 0.2% (w/v).

In embodiment 41, the pharmaceutical composition of any of embodiments 1-40 is provided, further comprising an aluminum adjuvant.

In embodiment 42, a pharmaceutical composition is provided comprising virus-like particles (VLPs) of at least one type of human papillomavirus (HPV), wherein the at least one type of HPV is selected from the group consisting of HPV types: 6, 11, 16, 18, 26, 31, 33, 35, 39, 45, 51, 52, 53, 55, 56, 58, 59, 66, 68, 73, and 82; and a chitosan adjuvant including 0.1 μg to about 50 mg of a chitosan, wherein the HPV VLPs comprise recombinant HPV L1 or recombinant HPV L1+L2 protein, wherein the HPV VLPs of each of the at least one HPV types are present in a concentration of about 10 μg to about 300 μg per 0.5 mL of the pharmaceutical composition, and wherein the total VLP concentration is between 10 μg and 2000 μg per 0.5 mL of the pharmaceutical composition.

In embodiment 43, the pharmaceutical composition of embodiment 42 is provided, wherein the composition further comprises about 100 μg to about 3500 μg of an aluminum adjuvant.

In embodiment 44, the pharmaceutical composition of embodiment 43 is provided, wherein the HPV VLPs are adsorbed onto the aluminum adjuvant.

In embodiment 45, the pharmaceutical composition of any of embodiments 42-44 is provided, wherein the composition comprises VLPs of HPV types 16 and 18.

In embodiment 46, the pharmaceutical composition of any of embodiments 42-45 is provided, wherein the composition comprises VLPs of HPV types 6, 11, 16, and 18.

In embodiment 47, the pharmaceutical composition of any of embodiments 42-46 is provided, wherein the composition comprises VLPs of HPV types 31, 45, 52, and 58.

In embodiment 48, the pharmaceutical composition of any of embodiments 42-47 is provided, wherein the composition comprises VLPs of HPV types 6, 11, 16, 18, 31, 33, 45, 52, and 58.

In embodiment 49, the pharmaceutical composition of any of embodiments 42-48 is provided, wherein the composition further comprises a buffer.

In embodiment 50, the pharmaceutical composition of any of embodiments 42-49 is provided, wherein the pharmaceutical composition further comprises a salt.

In embodiment 51, the pharmaceutical composition of any of embodiments 42-50 is provided, wherein the chitosan is a water soluble chitosan.

In embodiment 52, the pharmaceutical composition of any of embodiments 42-51 is provided, wherein the chitosan is an acid soluble chitosan.

In embodiment 53, the pharmaceutical composition of any of embodiments 42-52 is provided, wherein the composition is made by mixing an HPV vaccine and a chitosan adjuvant; wherein the HPV vaccine comprises HPV VLPs and a pharmaceutically acceptable carrier and the chitosan adjuvant comprises a chitosan and a pharmaceutically acceptable carrier.

In embodiment 54, the pharmaceutical composition of embodiments 53 is provided, wherein the chitosan adjuvant comprises a water-soluble chitosan having a deacetylation of 75% or greater.

In embodiment 55, the pharmaceutical composition of any of embodiments 53-54 is provided, wherein the chitosan adjuvant comprises a water-soluble chitosan having a viscosity in the range of about 1 cP to about 200 cP when measured with a viscosimeter at 20° C. at a standard concentration.

In embodiment 56, the pharmaceutical composition of any of embodiments 53-55 is provided, wherein the chitosan adjuvant comprises a water-soluble chitosan having a deacetylation of 85%-99%.

In embodiment 57, the pharmaceutical composition of any of embodiments 53-56 is provided, wherein the chitosan adjuvant comprises a water-soluble chitosan having a viscosity in the range of about 5 cP to about 10 cP when measured with a viscosimeter at 20° C. at a standard concentration.

In embodiment 58, the pharmaceutical composition of any of embodiments 53-57 is provided, wherein the chitosan adjuvant comprises a water-soluble chitosan having a deacetylation greater than 90% and a viscosity in the range of about 5 cP to about 10 cP when measured with a viscosimeter at 20° C. at a standard concentration.

In embodiment 59, the pharmaceutical composition of any of embodiments 53-58 is provided, wherein the chitosan adjuvant comprises a water-soluble chitosan having a deacetylation greater than 95% and a viscosity of less than 5 cP when measured with a viscosimeter at 20° C. at a standard concentration.

In embodiment 60, the pharmaceutical composition of embodiment 53 is provided, wherein the chitosan adjuvant comprises an acid-soluble chitosan having a deacetylation of 75% or greater.

In embodiment 61, the pharmaceutical composition of any of embodiments 53 or 60 is provided, wherein the chitosan adjuvant comprises an acid-soluble chitosan having a viscosity in the range of about 1 cP to about 200 cP when measured with a viscosimeter at 20° C. at a standard concentration.

In embodiment 62, the pharmaceutical composition of any of embodiments 53 or 60-61 is provided, wherein the chitosan adjuvant comprises an acid-soluble chitosan having a deacetylation of 85%-99%.

In embodiment 63, the pharmaceutical composition of any of embodiments 53 or 60-62 is provided, wherein the chitosan adjuvant comprises an acid-soluble chitosan having a viscosity in the range of about 5 cP to about 10 cP when measured with a viscosimeter at 20° C. at a standard concentration.

In embodiment 64, the pharmaceutical composition of any of embodiments 53 or 60-63 is provided, wherein the chitosan adjuvant comprises an acid-soluble chitosan having a deacetylation greater than 90% and a viscosity in the range of about 5 cP to about 10 cP when measured with a viscosimeter at 20° C. at a standard concentration.

In embodiment 65, the pharmaceutical composition of any of embodiments 53 or 60-63 is provided, wherein the chitosan adjuvant comprises an acid-soluble chitosan having a deacetylation greater than 95% and a viscosity of less than 5 cP when measured with a viscosimeter at 20° C. at a standard concentration.

In embodiment 66, the pharmaceutical composition of any of embodiments 53-65 is provided, wherein the chitosan adjuvant further comprises a buffer.

In embodiment 67, the pharmaceutical composition of embodiment 66 is provided, wherein the buffer is selected from the group consisting of: acetic acid, histidine, citrate, Bis-Tris, HEPES, phosphate, MES, sodium chloride, and combinations thereof.

In embodiment 68, the pharmaceutical composition of any of embodiments 66 and 67 is provided, wherein the buffer is present in the amount of about 1 mMol to about 100 mMol.

In embodiment 69, the pharmaceutical composition of any of embodiments 53-68 is provided, wherein the chitosan adjuvant further comprises a tonicity modifier.

In embodiment 70, the pharmaceutical composition of embodiment 69 is provided, wherein the tonicity modifier is selected from the group consisting of: sodium chloride, potassium chloride, sucrose, trehalose and combinations thereof.

In embodiment 71, the pharmaceutical composition of any of embodiments 69 and 70 is provided, wherein the tonicity modifier is present in the amount of about 10 mMol to about 500 mMol.

In embodiment 72, the pharmaceutical composition of any of embodiments 53-71 is provided, wherein the chitosan adjuvant further comprises a detergent.

In embodiment 73, the pharmaceutical composition of embodiment 72 is provided, wherein the detergent is selected from the group consisting of Polysorbate 80, Polysorbate 20, Poloxamer 188, and combinations thereof.

In embodiment 74, the pharmaceutical composition of any of embodiments 72 and 73 is provided, wherein the detergent is present in the amount of about 0.001% (w/v) to about 0.2% (w/v).

In embodiment 75, a single-dose vaccine composition is provided that includes a chitosan, virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, and a pharmaceutically acceptable carrier; wherein the single-dose vaccine composition provides an elevated or comparable anti-HPV immune response relative to multiple doses of the same composition formulated without a chitosan adjuvant.

In embodiment 76, the pharmaceutical composition of embodiment 75 is provided, wherein the vaccine further comprises an aluminum adjuvant.

In embodiment 77, the pharmaceutical composition of embodiment 76 is provided, wherein the HPV VLPs are adsorbed onto the aluminum adjuvant.

In embodiment 78, the pharmaceutical composition of any of embodiments 75-77 is provided, wherein each of the HPV VLPs are present in a concentration of about 10 μg to about 300 μg per 0.5 mL of the pharmaceutical composition and wherein the total HPV VLP concentration is between 10 μg and 2000 μg per 0.5 mL of the pharmaceutical composition.

In embodiment 79, the pharmaceutical composition of any of embodiments 1-74 or the single-dose vaccine composition of embodiments 75-78 is provided, wherein the HPV VLPs comprise HPV L1 protein and do not comprise HPV L2 protein.

In embodiment 80, the pharmaceutical composition of any of embodiments 1-66 or the single-dose vaccine composition of embodiments 75-78 is provided, wherein the HPV VLPs consist of HPV L1 protein.

In embodiment 81, a method of inducing an immune response to a human papillomavirus (HPV) in a human patient is provided comprising administering to the patient any of embodiments 1-74 or the single-dose vaccine composition of any of embodiments 75-80.

In embodiment 82, a method of inducing an immune response to a human papillomavirus (HPV) in a human patient is provided comprising co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan adjuvant

In embodiment 83, a method of preventing infection of a human patient by a human papillomavirus (HPV) is provided comprising administration to the patient the pharmaceutical composition of any of embodiments 1-74 or the single-dose vaccine composition of any of embodiments 75-80.

In embodiment 84, a method of preventing infection of a human patient by a human papillomavirus (HPV) is provided comprising co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan adjuvant.

In embodiment 85, a kit is provided comprising: (a) a human papilloma virus (HPV) vaccine; and (b) a chitosan adjuvant.

In embodiment 86, the pharmaceutical composition of embodiment 85 is provided, further comprising instructions for administering to a human patient the HPV vaccine and the chitosan adjuvant.

In embodiment 87, the pharmaceutical composition of any of embodiments 85-86 is provided, wherein the HPV vaccine comprises virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

In embodiment 88, a method of delivering a pharmaceutical composition to a subject that induces a neutralizing titer against an HPV antigen in the subject is provided comprising: administering to the subject a pharmaceutical composition comprising: a chitosan adjuvant, and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, whereby the administration of the pharmaceutical composition induces a neutralizing titer against the HPV antigen in the subject, wherein a single dose of the pharmaceutical composition provides enhanced or comparable neutralizing titers when compared to multiple doses of the same pharmaceutical composition when the same composition is formulated without a chitosan adjuvant.

In embodiment 89, the pharmaceutical composition of embodiment 88 is provided, wherein the pharmaceutical composition further comprises an aluminum adjuvant.

### EXAMPLES

### Example 1: Preparation of an Acid-Soluble Chitosan Adjuvant

Compositions that include a chitosan adjuvant of the present invention were made according to the following method. Approximately 12 mL of 1% acetic acid was placed in a 25 mL volumetric flask. Approximately 0.903 g of chitosan acquired from Sigma-Aldrich (product number 448869) was added to the flask. The chitosan and acetic acid mixture was stirred until the chitosan was dissolved. 10 mM histidine was added to the chitosan solution. The pH of the resulting solution was adjusted to approximately 5.7 using 20% w/v sodium hydroxide solution. Q.S. water was then added. The resulting solution was sterile filtered in a sterile biosafety cabinet.

### Example 2: Preparation of a Water-Soluble Chitosan Adjuvant

Compositions that include a chitosan adjuvant of the present invention were made according to the following method. Approximately 600 mg grams of chitosan hydrochloride (HMC Item #54047) having a deacetylation of 96.5% and viscosity of 18 was added to 9.4 mL of water and then combined with an equal volume of 10 mM histidine 325 mM NaCl solution having a pH of 6.2. The contents were mixed until the chitosan hydrochloride solids were dissolved. Multiple solutions were made, each had a pH of the resulting solution between 5.6 to 5.9. The resulting solution was sterile filtered in a sterile biosafety cabinet.

### Example 3: Preparation of a Water-Soluble Chitosan Adjuvant

Compositions that include a chitosan adjuvant of the present invention were made according to the following method. Approximately 12.5 mL of water was placed in a 25 mL volumetric flask. Approximately 0.04 grams of histidine was then added to the water. The solution was stirred until the solids were dissolved. Approximately 300 mg of chitosan hydrochloride (Heppe Medical Chitosan “HMC” Item #54046) having a deacetylation of 95.4% and viscosity of 97 was added to 12.5 mL of water and then combined with an equal volume of 10 mM histidine 325 mM NaCl at pH 6.2. The contents were mixed until the chitosan hydrochloride solids were dissolved. The flask was then filled to the volumetric line with Q.S. HPLC water to a target volume of 25 mL. Multiple solutions were made; the pH of the resulting solutions were between 5.6 to 5.9. The resulting solution was sterile filtered in a sterile biosafety cabinet.

### Example 4: Immunogenicity and Durability of a Single Dose of 9vHPV Vaccine+Water-Soluble Chitosan Adjuvant in Rabbits

In this example, a 9 valent HPV/aluminum adjuvant vaccine that included the capsid (L1) protein of HPV types 6, 11, 16, 18, 31, 33, 45, 52, and 58 (hereinafter “9vHPV Vaccine”) 9vHPV Vaccine (the 9V vaccine further contained aluminum (provided as AAHS), sodium chloride, L-histidine, and polysorbate 80) was combined with the Chitosan Adjuvant of Example 1. The immunogenicity was then evaluated in a rabbit preclinical immunogenicity model. As described in Table II, three groups, each including 4 New Zealand white rabbits, were injected intramuscularly with a single-dose regimen (i.e., one dose administered at week 0) or a multi-dose regimen (i.e., one dose administered at week 0 and a second dose administered at week 4) with 9vHPV Vaccine and compared to a group that received a single-dose (i.e., one dose administered at week 0) of 9vHPV Vaccine plus the water-soluble chitosan adjuvant of Example 1. The 0.5 mL inoculums of the 9vHPV Vaccine plus the Chitosan Adjuvant of Example 1 were prepared by admixing the 9vHPV Vaccine with 4 mg the water-soluble chitosan adjuvant of Example 1 and administered by intramuscular injection into the rabbit hind quadricep within 4 hours.

TABLE II
Groups, Dose Levels, and Dosing Schedule
in Rabbits for Study SD-HPV-004

| | No. of | | Chitosan | | Dosing |
|---|---|---|---|---|---|
| Group | Rabbits | Inoculum | Dose | ROA^{a} | Schedule |
| 1 | 4 | 9vHPV Vaccine ^{b} | NA | IM | week 0 |
| 2 | 4 | 9vHPV Vaccine | NA | IM | 0, 4 weeks |
| 3 | 4 | 9vHPV Vaccine + | 4 mg | IM | week 0 |
| | | Chitosan adjuvant of | | | |
| | | Example 1 | | | |

^{a}All doses were delivered in 500 μL to single quadriceps
^{b} Rabbit dose of a 9vHPV Vaccine is equivalent to a 1/20 human dose
IM = intramuscular;
NA = not applicable;
ROA = route of administration

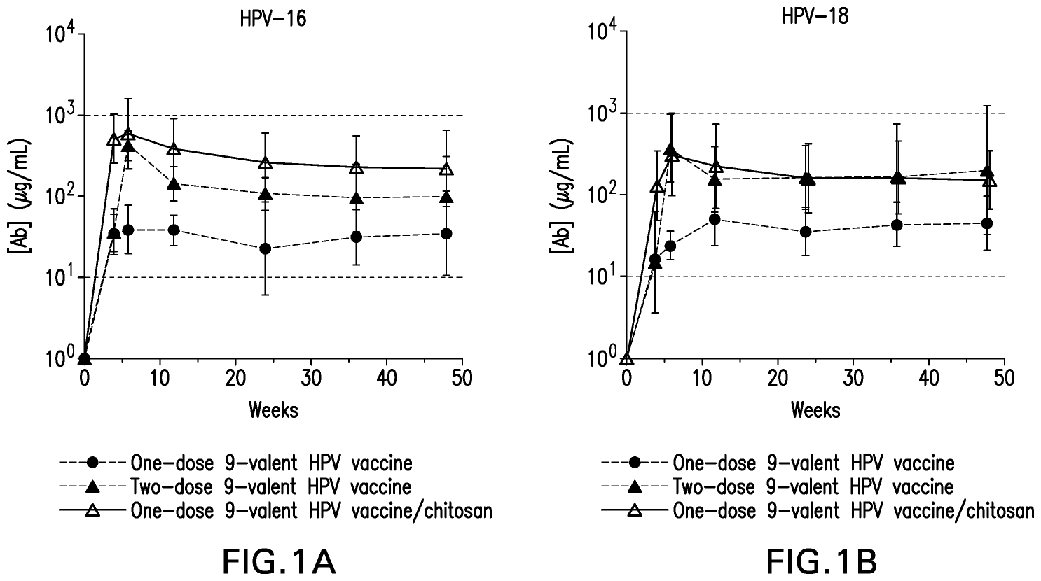

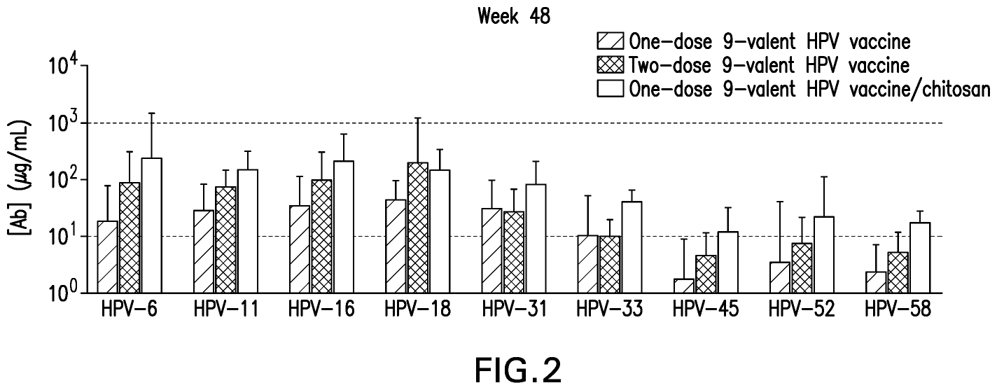

To assess immunogenicity, samples of serum from individual animals were evaluated using a multiplex assay that detects antibody levels to all 9 HPV types in the 9vHPV Vaccine. VLP-specific HPV antibody concentrations were determined at study week 4, 6, 12, 24, 36 and 48. Titers at week 0 were set at 1 μg/ml, based on analysis of sera from naive rabbits. Representative titers to HPV VLP-16 and HPV VLP-18 are shown in FIGS. 1A and 1B, respectively. A single inoculation of the Chitosan Adjuvant of Example 1 combined with 9vHPV Vaccine induced similar antibody concentrations to two doses of a 9vHPV Vaccine injected 4 weeks apart. Antibody levels were approximately a log higher in animals that received the 9vHPV Vaccine plus the Chitosan Adjuvant of Example 1 than in those receiving a single dose of a 9vHPV Vaccine that did not include the Chitosan Adjuvant of Example 1. The high antibody levels in the 9vHPV Vaccine plus the Chitosan Adjuvant of Example 1 group remained mostly unchanged throughout the duration of the study (48 weeks). Immune responses observed for all 9 VLP types at week 48 are presented in FIG. 2. The data are presented as geometric mean concentrations and 95% confidence intervals (CI). Anti-HPV antibody levels of the group of rabbits treated with a single-dose of the 9vHPV Vaccine plus the Chitosan Adjuvant of Example 1 were similar to or higher than the anti-HPV antibody levels of the group of rabbits treated with two-doses of the 9vHPV Vaccine group for all VLP types.

### Example 5: Immunogenicity and Durability of 9vHPV Vaccine+Chitosan Adjuvant of Example 1 in Rhesus Macaques

In this example, a 9 valent HPV/aluminum adjuvant vaccine that included the capsid (L1) protein of HPV types 6, 11, 16, 18, 31, 33, 45, 52, and 58 (hereinafter “9vHPV Vaccine”) 9vHPV Vaccine (the 9V vaccine further contained aluminum (provided as AAHS), sodium chloride, L-histidine, and polysorbate 80) was combined with increasing doses of the Chitosan Adjuvant of Example 1 (1, 4, or 12 mg; sourced from Sigma) The immunogenicity was then evaluated in rhesus macaques. As described in Table III, five groups, each containing 6 rhesus macaques, were injected intramuscularly with a single-dose regimen (i.e., one dose administered at week 0) or a multi-dose regimen (i.e., one dose administered at week 0 and a second dose administered at week 4) with 9vHPV Vaccine and compared to a group that received a single-dose (i.e., one dose administered at week 0) of 9vHPV Vaccine plus the water-soluble chitosan adjuvant of Example 1.

The 1.0 mL inoculums were prepared by mixing a 9vHPV Vaccine and 1, 4, or 12 mg of the Chitosan Adjuvant of Example 1 and administered into the rhesus macaque quadricep within 4 hours.

TABLE III
Groups, Dose Levels, and Dosing Schedule in
Non-Human Primates for Study SD-HPV-009

| | No. of | | | | | |
|---|---|---|---|---|---|---|
| | Rhesus | | Chitosan | | | Dosing |
| Group | Macaques | Inoculum | Dose | | ROA^{a} | Schedule |
| 1 | 6 | 9vHPV Vaccine ^{b} | NA | | IM | 0, 4 weeks |
| 2 | 6 | 9vHPV Vaccine | NA | | IM | week 0 |
| 3 | 6 | 9vHPV Vaccine + | 1 | mg | IM | week 0 |
| | | Chitosan Adjuvant of | | | | |
| | | Example 1 | | | | |
| 4 | 6 | 9vHPV Vaccine + | 4 | mg | IM | week 0 |
| | | Chitosan Adjuvant of | | | | |
| | | Example 1 | | | | |
| 5 | 6 | 9vHPV Vaccine + | 12 | mg | IM | week 0 |
| | | Chitosan Adjuvant of | | | | |
| | | Example 1 | | | | |

^{a}All doses were delivered in 1 mL to single quadriceps
^{b} Rhesus monkey dose of a 9vHPV Vaccine is equivalent to a 1/20 human dose
IM = intramuscular;
NA = not applicable;
ROA = route of administration

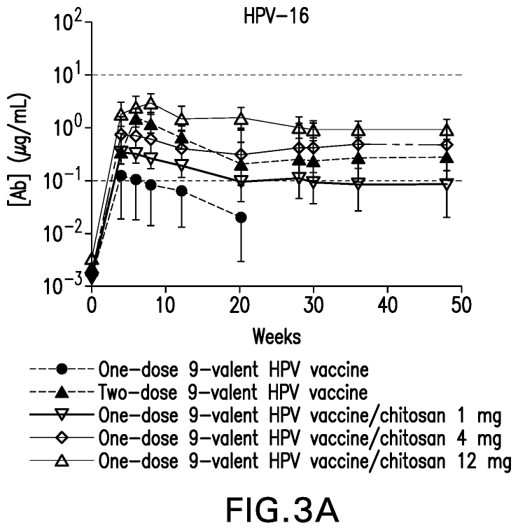

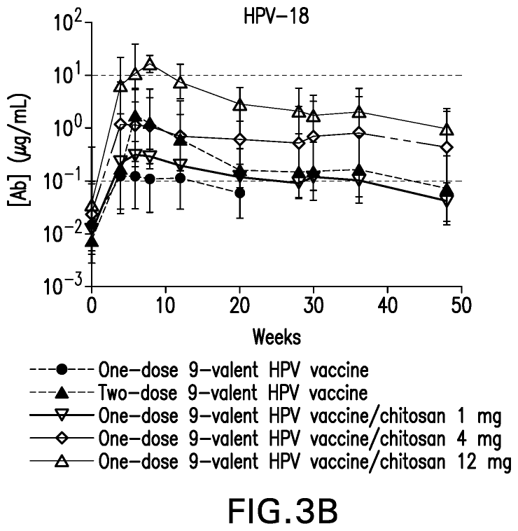

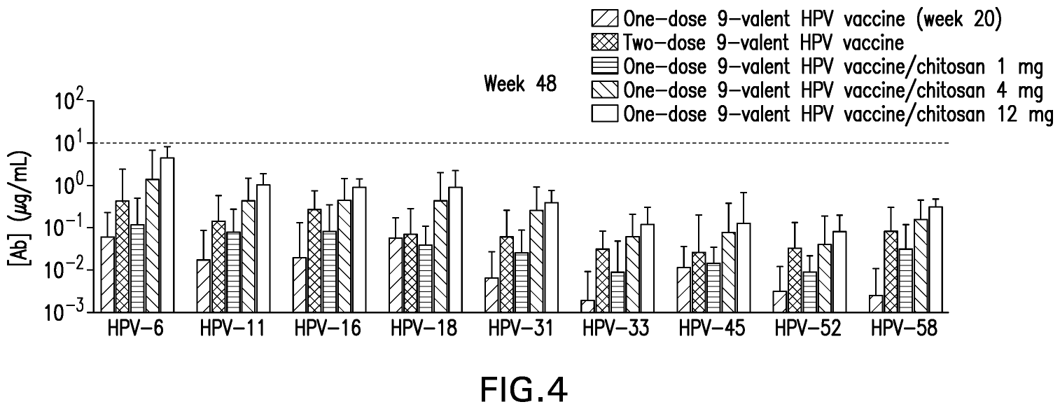

To assess immunogenicity, samples of sera from individual animals were evaluated using a multiplex assay for antibody levels to all 9 HPV types in a 9vHPV Vaccine. VLP-specific HPV antibody concentrations were determined at study week 0, 4, 6, 8, 12, 20, 28, 30, 36 and 48. Representative titers to HPV VLP-16 and HPV VLP-18 are shown in FIGS. 3A and 3B. The results verified that adjuvating effect of chitosan on HPV antibody titers was dose dependent. Animals inoculated with a 9vHPV Vaccine combined with a chitosan adjuvant that included 1 mg, 4 mg, or 12 mg of chitosan yielded HPV antibody titers higher than the single-dose a 9vHPV Vaccine group at all timepoints tested. Antibody levels trended lower than the two-dose a 9vHPV Vaccine group in the animals that received a vaccine that included a chitosan adjuvant having 1 mg chitosan, but titers were comparable or higher in the group that included a chitosan adjuvant having 4 mg and a chitosan adjuvant having 12 mg chitosan. These titers remained higher than in the two-dose control group throughout the end of the study (48 weeks). As shown in FIG. 4, these findings apply to all 9 VLP types included in a 9vHPV Vaccine.

### Example 6: Immunogenicity and Durability of a Single Dose of a 9vHPV Vaccine+Water-Soluble Chitosan Adjuvant in Rhesus Macaques

A 9 valent HPV/aluminum adjuvant vaccine that included the capsid (L1) protein of HPV types 6, 11, 16, 18, 31, 33, 45, 52, and 58 (hereinafter “9vHPV Vaccine”) (the 9V Vaccine further contained aluminum (provided as AAHS), sodium chloride, L-histidine, and polysorbate 80) was combined with two chitosan products with different acetylation and viscosity levels (Chitosan Adjuvant of Example 2 and Chitosan Adjuvant of Example 3) sourced from Heppe Medical Chitosan (HMC). Increasing doses of chitosan adjuvant (2, 6, or 16 mg of Chitosan Adjuvant of Example 2 and 2 or 6 mg of Chitosan Adjuvant of Example 3) were evaluated. The highest dose of each chitosan selected for testing was limited by the filterability of the resulting formulation due to the viscosity of the particular chitosan used, i.e., Lot #54047 vs. Lot #54046. The group designations are described in Table IV. The immunogenicity was evaluated in rhesus macaques. Six groups of 4 or 5 rhesus macaques each, were inoculated with either a 9vHPV Vaccine alone or a 9vHPV Vaccine mixed with the Chitosan Adjuvant of Examples 2 and 3. Animals in group 1 were given a second dose of a 9vHPV Vaccine after four weeks, while none of the other groups were boosted. The 1.0 mL inoculums were prepared by mixing a 9vHPV Vaccine and chitosan adjuvant of either Example 2 or Example 3 before administration into the rhesus macaque quadricep within 1 hour of formulation.

TABLE IV
Groups, Dose Levels, and Dosing Schedule in
Non-human Primates for Study SD-HPV-036

| | No. of | | | | | |
|---|---|---|---|---|---|---|
| | Rhesus | | Chitosan | | | Dosing |
| Group | Macaques | Inoculum | Dose | | ROA^{a} | Schedule |
| 1 | 4 | 9vHPV Vaccine ^{b} | NA | | IM | 0, 4 weeks |
| 2 | 5 | 9vHPV Vaccine + | 2 | mg | IM | week 0 |
| | | Chitosan Adjuvant of | | | | |
| | | Example 2 | | | | |
| 3 | 5 | 9vHPV Vaccine + | 6 | mg | IM | week 0 |
| | | Chitosan Adjuvant of | | | | |
| | | Example 2 | | | | |
| 4 | 5 | 9vHPV Vaccine + | 16 | mg | IM | week 0 |
| | | Chitosan Adjuvant of | | | | |
| | | Example 2 | | | | |
| 5 | 5 | 9vHPV Vaccine + | 2 | mg | IM | week 0 |
| | | Chitosan Adjuvant of | | | | |
| | | Example 3 | | | | |
| 6 | 5 | 9vHPV Vaccine + | 6 | mg | IM | week 0 |
| | | Chitosan Adjuvant of | | | | |
| | | Example 3 | | | | |

^{a}All doses were delivered in 1 mL to single quadriceps
^{b} Rhesus monkey dose of 9vHPV Vaccine is equivalent to a 1/20 human dose
IM = intramuscular;
NA = not applicable;
ROA = route of administration

Rhesus macaques (n=4 or 5/group) were inoculated with either 9vHPV Vaccine alone or 9vHPV Vaccine mixed with chitosan adjuvant. Animals in group 1 were given a second dose of 9vHPV Vaccine (two-dose G9) after four weeks, while none of the other groups were boosted.

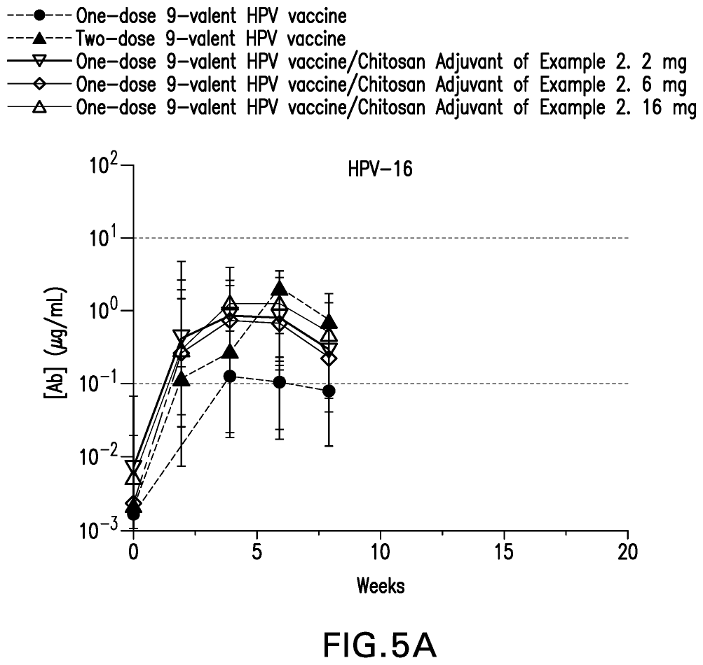

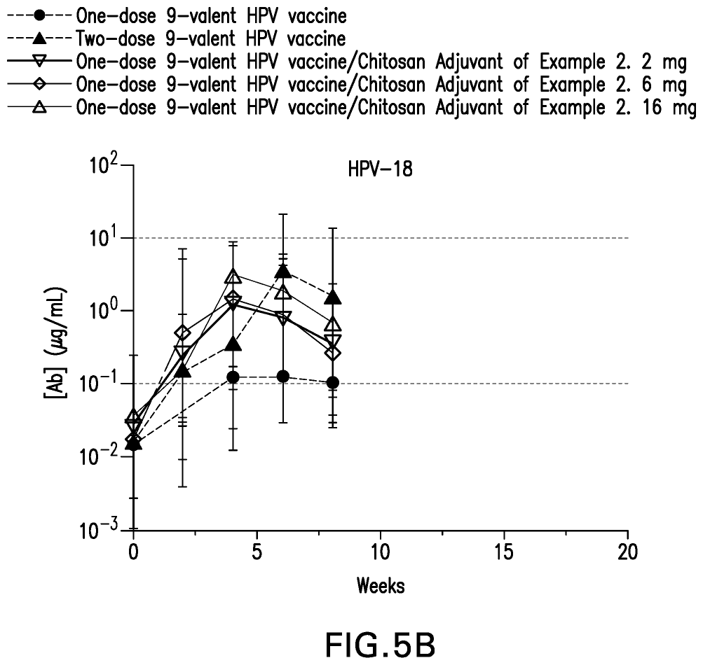

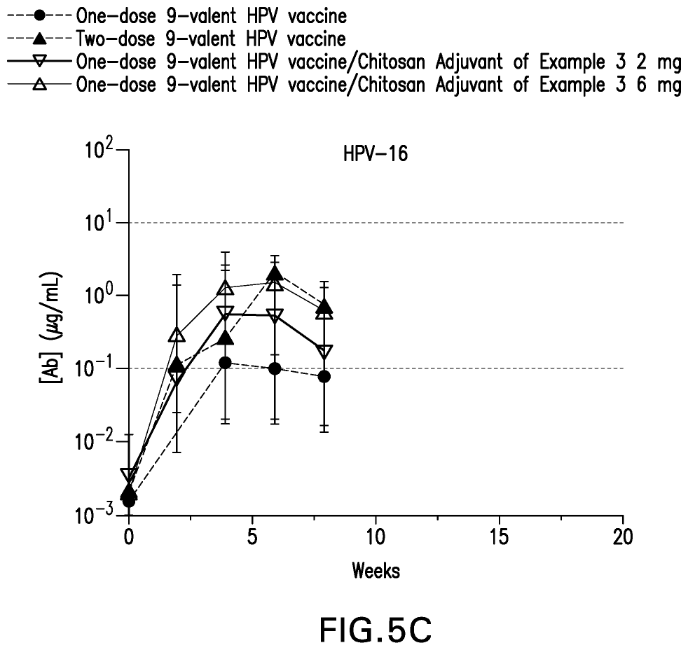

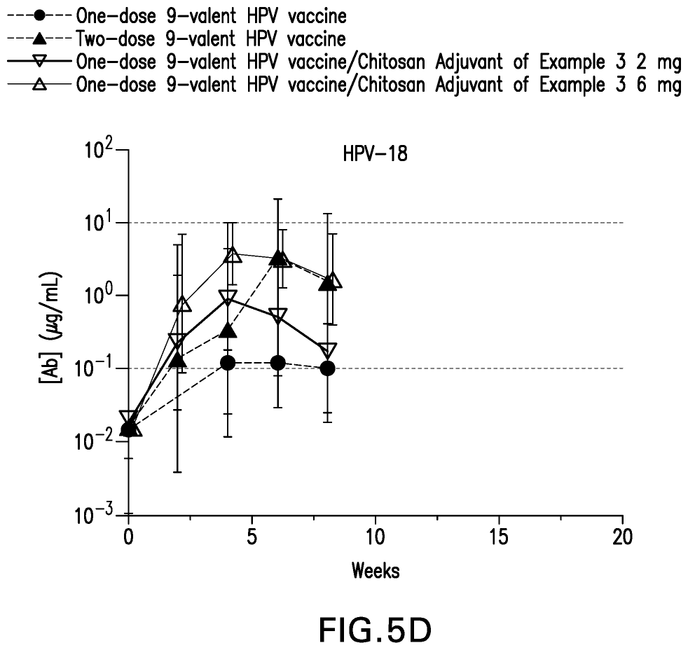

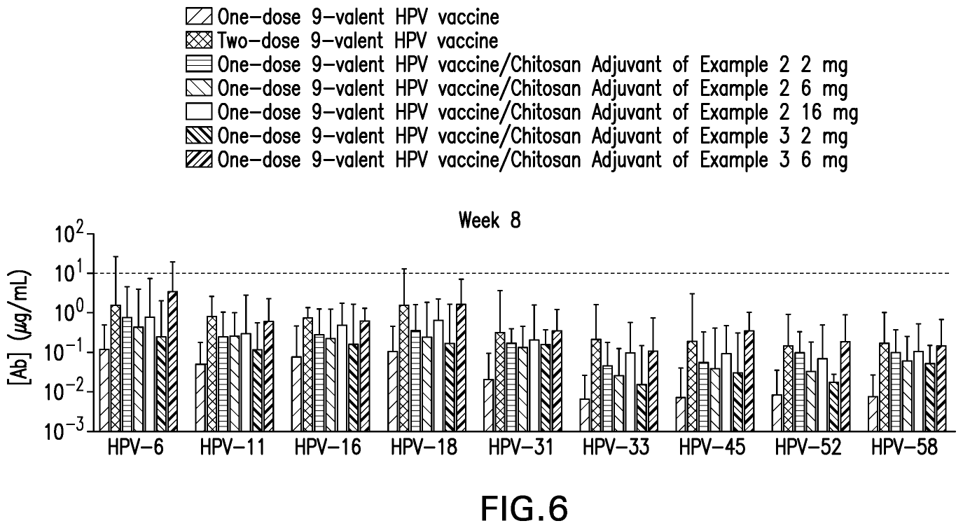

To assess immunogenicity, sera from individual animals are being evaluated using a multiplex assay that detects antibody levels to all 9 HPV types in a 9vHPV Vaccine. VLP-specific HPV antibody concentrations were determined at study week 2, 4, 6, 8, 12, 20, 30 and 40. Representative titers to HPV VLP-16 and HPV VLP-18 are shown in FIGS. 5A, 5B, 5C, and 5D. Both Chitosan Adjuvant of Example 2 (FIGS. 5A and 5B) and Chitosan Adjuvant of Example 3 (FIGS. 5C and 5D) combined with a 9vHPV Vaccine induced higher responses than a single-dose of a 9vHPV Vaccine without a chitosan adjuvant. The adjuvating effect appears to be dose dependent for both Chitosan Adjuvant of Example 2 and Chitosan Adjuvant of Example 3. Interestingly, while the higher viscosity of chitosan used in Chitosan Adjuvant of Example 2 limited dosing to 6 mg/animal, its adjuvant effect was at least as good as the highest dose (16 mg) of the Chitosan Adjuvant of Example 3. The responses measured at study week 40 are comparable to what is achieved in the two dose 9vHPV Vaccine group for all 9 VLP types (FIG. 6).

What is claimed:

1. A pharmaceutical composition comprising:
virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82,
a chitosan; and
a pharmaceutically acceptable carrier.

2. The pharmaceutical composition of claim 1, wherein the composition comprises VLPs of HPV types 16 and 18.

3. The pharmaceutical composition of claim 1, wherein the composition comprises VLPs of HPV types 6, 11, 16, and 18.

4. The pharmaceutical composition of claim 1, wherein the composition comprises VLPs of HPV types 31, 45, 52, and 58.

5. The pharmaceutical composition of claim 1, wherein the composition comprises VLPs of the following HPV types:
(a) 6, 11, 16, 18, 31, 33, 45, 52, and 58;
(b) 6, 11, 16, 18, 31, 33, 35, 45, 52, and 58;
(c) 6, 11, 16, 18, 31, 33, 35, 45, 52, 58, and 59;
(d) 6, 11, 16, 18, 31, 33, 45, 52, 58, 59, and 68;
(e) 6, 11, 16, 18, 31, 33, 35, 39, 45, 51, 52, 56, 58, and 59;
(f) 6, 11, 16, 18, 26, 31, 33, 35, 45, 51, 52, 58, 59, and 69;
(g) 6, 11, 16, 18, 26, 31, 33, 35, 39, 45, 51, 52, 58, 59, 68, 69, and 70; or
(h) 6, 11, 16, 18, 26, 31, 33, 35, 39, 45, 51, 52, 53, 56, 58, 59, 66, 68, 69, and 70.

6. The pharmaceutical composition of claim 1, wherein the pharmaceutical composition further comprises a buffer.

7. The pharmaceutical composition of claim 1, wherein the pharmaceutical composition further comprises aluminum.

8. The pharmaceutical composition of claim 1, wherein the composition is made by mixing an HPV vaccine and a chitosan adjuvant; wherein the HPV vaccine comprises HPV VLPs and a pharmaceutically acceptable carrier and the chitosan adjuvant comprises a chitosan and a pharmaceutically acceptable carrier.

9. The pharmaceutical composition of claim 1, wherein the composition is made by mixing an HPV vaccine and chitosan.

10. The pharmaceutical composition of claim 8, wherein the chitosan adjuvant comprises a water-soluble chitosan having a viscosity in the range of about 1 cP to about 200 cP when measured with a viscosimeter at 20° C. at a standard concentration.

11. The pharmaceutical composition of claim 8, wherein the chitosan adjuvant comprises a water-soluble chitosan having a deacetylation of 85%-99%.

12. The pharmaceutical composition of claim 8, wherein the chitosan adjuvant comprises a water-soluble chitosan having a deacetylation greater than 90% and a viscosity in the range of about 5 cP to about 10 cP when measured with a viscosimeter at 20° C. at a standard concentration.

13. The pharmaceutical composition of claim 8, wherein the chitosan adjuvant comprises a water-soluble chitosan having a deacetylation greater than 95% and a viscosity of less than 5 cP when measured with a viscosimeter at 20° C. at a standard concentration.

14. The pharmaceutical composition of claim 8, wherein the chitosan adjuvant comprises an acid-soluble chitosan having a viscosity in the range of about 1 cP to about 200 cP when measured with a viscosimeter at 20° C. at a standard concentration and a deacetylation of 85%-99%.

15. The pharmaceutical composition of claim 8, wherein the chitosan adjuvant comprises an acid-soluble chitosan having a deacetylation greater than 95% and a viscosity of less than 5 cP when measured with a viscosimeter at 20° C. at a standard concentration.

16. The pharmaceutical composition of claim 8, wherein the chitosan adjuvant further comprises a buffer selected from the group consisting of: acetic acid, histidine, citrate, Bis-Tris, HEPES, phosphate, MES, sodium chloride, and combinations thereof.

17. The pharmaceutical composition of claim 8, wherein the chitosan adjuvant further comprises a tonicity modifier selected from the group consisting of: sodium chloride, potassium chloride, sucrose, trehalose and combinations thereof.

18. The pharmaceutical composition of claim 8, wherein the chitosan adjuvant further comprises a detergent selected from the group consisting of Polysorbate 80, Polysorbate 20, Poloxamer 188, and combinations thereof.

19. A pharmaceutical composition comprising
virus-like particles (VLPs) of at least one type of human papillomavirus (HPV), wherein the at least one type of HPV is selected from the group consisting of HPV types: 6, 11, 16, 18, 26, 31, 33, 35, 39, 45, 51, 52, 53, 55, 56, 58, 59, 66, 68, 73, and 82; and
0.1 μg to about 50 mg of a chitosan,
wherein the HPV VLPs comprise recombinant HPV L1 or recombinant HPV L1+L2 protein,
wherein the HPV VLPs of each of the at least one HPV types are present in a concentration of about 10 μg to about 300 μg per 0.5 mL of the pharmaceutical composition, and
wherein the total VLP concentration is between 10 μg and 2000 μs per 0.5 mL of the pharmaceutical composition.

20. The pharmaceutical composition of claim 19, further comprising about 100 μg to about 3500 μg of an aluminum adjuvant.

21. The pharmaceutical composition of claim 19, wherein the composition comprises VLPs of the following HPV types:
(a) 6, 11, 16, 18, 31, 33, 45, 52, and 58;
(b) 6, 11, 16, 18, 31, 33, 35, 45, 52, and 58;
(c) 6, 11, 16, 18, 31, 33, 35, 45, 52, 58, and 59;
(d) 6, 11, 16, 18, 31, 33, 45, 52, 58, 59, and 68;
(e) 6, 11, 16, 18, 31, 33, 35, 39, 45, 51, 52, 56, 58, and 59;
(f) 6, 11, 16, 18, 26, 31, 33, 35, 45, 51, 52, 58, 59, and 69;
(g) 6, 11, 16, 18, 26, 31, 33, 35, 39, 45, 51, 52, 58, 59, 68, 69, and 70; or
(h) 6, 11, 16, 18, 26, 31, 33, 35, 39, 45, 51, 52, 53, 56, 58, 59, 66, 68, 69, and 70.

22. The pharmaceutical composition of claim 19, wherein the composition is made by mixing an HPV vaccine and a chitosan adjuvant; wherein the HPV vaccine comprises HPV VLPs and a pharmaceutically acceptable carrier and the chitosan adjuvant comprises a chitosan and a pharmaceutically acceptable carrier.

23. The pharmaceutical composition of claim 22, wherein the chitosan adjuvant comprises a water-soluble chitosan having a deacetylation greater than 90% and a viscosity in the range of about 5 cP to about 10 cP when measured with a viscosimeter at 20° C. at a standard concentration.

24. The pharmaceutical composition of claim 22, wherein the chitosan adjuvant comprises a water-soluble chitosan having a deacetylation greater than 95% and a viscosity of less than 5 cP when measured with a viscosimeter at 20° C. at a standard concentration.

25. The pharmaceutical composition of claim 22, wherein the chitosan adjuvant comprises an acid-soluble chitosan having a deacetylation greater than 90% and a viscosity in the range of about 5 cP to about 10 cP when measured with a viscosimeter at 20° C. at a standard concentration.

26. The pharmaceutical composition of claim 22, wherein the chitosan adjuvant comprises an acid-soluble chitosan having a deacetylation greater than 95% and a viscosity of less than 5 cP when measured with a viscosimeter at 20° C. at a standard concentration.

27. A single-dose vaccine composition comprising:
a chitosan,
virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, and
a pharmaceutically acceptable carrier;
wherein the single-dose vaccine composition provides an elevated or comparable anti-HPV immune response relative to multiple doses of the same composition formulated without a chitosan adjuvant.

28. A method of inducing an immune response to a human papillomavirus (HPV) in a human patient comprising administering to the patient the pharmaceutical composition of claim 1.

29. A method of inducing an immune response to a human papillomavirus (HPV) in a human patient comprising co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan.

30. A method of preventing infection of a human patient by a human papillomavirus (HPV) comprising administration to the patient the pharmaceutical composition of claim 1.

31. A kit comprising:
(a) a human papilloma virus (HPV) vaccine comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82; and
(b) a chitosan.

32. A method of delivering a pharmaceutical composition to a subject that induces a neutralizing titer against an HPV antigen in the subject comprising:
administering to the subject a pharmaceutical composition comprising:
a chitosan adjuvant, and
virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82,
whereby the administration of the pharmaceutical composition induces a neutralizing titer against the HPV antigen in the subject,
wherein a single dose of the pharmaceutical composition provides enhanced or comparable neutralizing titers when compared to multiple doses of the same pharmaceutical composition when the same composition is formulated without a chitosan adjuvant.
